(19) United States
(12) Patent Application Publication
(10) Pub. No.: US 2026/0263672 A1
(43) Pub. Date: Sep. 10, 2026

(54) Closed-Loop Peritoneal Dialysis System

(71) Applicant: Byonyks Medical Devices, Inc., Itasca, IL (US)

(72) Inventors: Rodney S. Kenley, Itasca, IL (US); Michael Wollowitz, Itasca, IL (US)

(21) Appl. No.: 19/165,536

(22) PCT Filed: Mar. 15, 2024
(86) PCT No.: PCT/US2024/020187

§ 371 Date: Sep. 15, 2025

Related U.S. Application Data
(60) Provisional application No. 63/490,676, filed on Mar. 16, 2023.

(51) Int. Cl. A61M 1/28; A61M 39/22

(52) U.S. Cl. CPC A61M 1/282; A61M 1/281; A61M 1/287; A61M 39/225

(57) ABSTRACT

A system includes a housing, a water supply line, a dialysate supply line, a drain line, and a patient line each extending from the housing. The system also includes a first port, a second port, a third port, and a fourth port that are accessible from outside the housing. The first port can connect to the water supply line, the second port can connect to the dialysate supply line, the third port can connect to the drain line, and the fourth port can connect to the patient line. The system also includes a first flush line, a second flush line, and a third flush line each contained within the housing. The first flush line terminates at the first port and the third port, the second flush line terminates at the second port, and the third flush line terminates at the fourth port and at the drain line within the housing.

### CROSS-REFERENCE TO RELATED APPLICATIONS

This application claims priority to U.S. Provisional Patent Application No. 63/490,676, filed on Mar. 16, 2023, the entire contents of which are incorporated by reference herein.

### BACKGROUND

There are two principal dialysis methods used to support patients requiring renal replacement therapy: hemodialysis and peritoneal dialysis. Peritoneal dialysis utilizes the patient's own peritoneum as a semipermeable membrane. The peritoneum is the membranous lining of the body cavity surrounding all of the organs between the diaphragm and the pelvis that, due to the large number of blood vessels and capillaries imbedded therein, is capable of acting as a natural semipermeable membrane.

In peritoneal dialysis, a sterile dialysate is infused into the peritoneal cavity by way of an indwelling catheter. This can be accomplished manually by gravity or with the use of a machine known as a cycler. An osmotic pressure gradient is generated by including an osmotic agent in the peritoneal dialysate. The osmotic agent used in the vast majority of peritoneal dialysate is glucose. The dialysate is allowed to dwell in the peritoneal cavity for a sufficient length of time (e.g. 4 hours) to yield a net removal of toxins and water after which the dialysate is drained and replaced with fresh dialysate.

There are two primary forms of peritoneal dialysis (PD): Continuous Ambulatory Peritoneal Dialysis (CAPD) and Continuous Cycling Peritoneal Dialysis (CCPD). With CCPD, fluid exchanges can be performed while the patient is sleeping, with the inflow and outflow of dialysate controlled by a cycler. This is designed to spare the patient from the drudgery of performing these exchanges during their waking hours. Typically, the cycler has left them with a final fill volume just before they wake up and this is left in the peritoneum until midday when the patient performs a drain. The patient then has the option to remain “dry” until that night which has the benefits of letting their peritoneal membrane recover from the constant onslaught of low pH/high osmolarity solution, or, if they require more toxin and water removal, they can instill another fill volume for the rest of the day.

One disadvantage of CCPD is the burden of storing, connecting, disconnecting, and disposing supplies that are used during the process, such as sterilized bags of solution, tubing sets and connectors, and the ancillary supplies required to perform aseptic connections/disconnections.

### SUMMARY

A first example includes a system for peritoneal dialysis, the system comprising: a housing; a water supply line, a dialysate supply line, a drain line, and a patient line each extending from the housing; a first port, a second port, a third port, and a fourth port that are accessible from outside the housing, wherein the first port is configured to connect to the water supply line, the second port is configured to connect to the dialysate supply line, the third port is configured to connect to the drain line, and the fourth port is configured to connect to the patient line; and a first flush line, a second flush line, and a third flush line each contained within the housing, wherein the first flush line terminates at the first port and at the third port, the second flush line terminates at the second port, and the third flush line terminates at the fourth port and at the drain line within the housing.

A second example includes a method of operating the system of the first example, the method comprising making a determination that: (i) the water supply line is not connected to the first port, (ii) the dialysate supply line is not connected to the second port, (iii) the drain line is not connected to the third port, and (iv) the patient line is connected to the fourth port; and in response to making the determination, operating the system in the priming mode.

A third example includes a method of operating the system of the first example, the method comprising making a determination that: (i) the water supply line is not connected to the first port, (ii) the dialysate supply line is not connected to the second port, (iii) the drain line is not connected to the third port, and (iv) the patient line is not connected to the fourth port; and in response to making the determination, operating the system in the patient drain mode.

A fourth example includes a method of operating the system of the first example, the method comprising making a determination that: (i) the water supply line is not connected to the first port, (ii) the dialysate supply line is not connected to the second port, (iii) the drain line is not connected to the third port, and (iv) the patient line is not connected to the fourth port; and in response to making the determination, operating the system in the patient fill mode.

A fifth example includes a method of operating the system of the first example, the method comprising making a determination that: (i) the water supply line is not connected to the first port, (ii) the dialysate supply line is connected to the second port, (iii) the drain line is not connected to the third port, and (iv) the patient line is connected to the fourth port; and in response to making the determination, operating the system in the flush mode.

A sixth example includes a method of operating the system of the first example, the method comprising making a determination that: (i) the water supply line is connected to the first port, (ii) the dialysate supply line is connected to the second port, (iii) the drain line is connected to the third port, and (iv) the patient line is connected to the fourth port; and in response to making the determination, operating the system in the disinfect mode.

A seventh example includes a method of operating the system of the first example, the method comprising making a determination that: (i) the disposable priming line is connected to the first port, (ii) the dialysate supply line is connected to the second port, (iii) the drain line is connected to the third port, and (iv) the patient line is connected to the fourth port; and in response to making the determination, operating the system in the air removal mode.

An eighth example includes a method of operating the system of the first example, the method comprising operating the system in the priming mode while the dialysate supply line is connected to a dialysate supply bag and the patient line is connected to the fourth port, thereby filling the system with the supply dialysate; operating the system in the patient drain mode while the patient line is connected to the patient's peritoneum, thereby draining the spent dialysate from the patient's peritoneum through the drain line; operating the system in the patient fill mode while the patient line is connected to the patient's peritoneum and the dialysate supply line is connected to the dialysate supply bag thereby filling the patient's peritoneum with the supply dialysate; operating the system in the flush mode while the water supply line is connected to the water supply bag, the dialysate supply line is connected to the second port, and the patient line is connected to the fourth port, thereby filling the system with water; operating the system in the disinfect mode while the water supply line is connected to the first port, the dialysate supply line is connected to the second port, the drain line is connected to the third port, and the patient line is connected to the fourth port, thereby circulating the heated water through the first flush line, the second flush line, and the third flush line; and operating the system in the air removal mode while the water supply line is connected to a first bag port of the water supply bag, the dialysate supply line is connected to the second port, the disposable priming line is connected to the first port and a second bag port of the water supply bag, the drain line is connected to the third port, and the patient line is connected to the fourth port, thereby trapping air received from the disposable priming line in the water supply bag.

When the term “substantially” or “about” is used herein, it is meant that the recited characteristic, parameter, or value need not be achieved exactly, but that deviations or variations, including, for example, tolerances, measurement error, measurement accuracy limitations, and other factors known to those of skill in the art may occur in amounts that do not preclude the effect the characteristic was intended to provide. In some examples disclosed herein, “substantially” or “about” means within +/−0-5% of the recited value.

These, as well as other aspects, advantages, and alternatives will become apparent to those of ordinary skill in the art by reading the following detailed description, with reference where appropriate to the accompanying drawings. Further, it should be understood that this summary and other descriptions and figures provided herein are intended to illustrate the invention by way of example only and, as such, that numerous variations are possible.

### BRIEF DESCRIPTION OF THE DRAWINGS

FIG. 1 is a block diagram of a system that includes a computing device, according to an example.

FIG. 2 is a schematic diagram of a system configured for operation in a priming mode, according to an example.

FIG. 3 is a schematic diagram of a system configured for operation in a patient drain mode or a patient fill mode, according to an example.

FIG. 4 is a schematic diagram of a system configured for operation in a flush mode, according to an example.

FIG. 5 is a schematic diagram of a system configured for operation in a disinfect mode, according to an example.

FIG. 6 is a schematic diagram of a system configured for operation in an air removal mode, according to an example.

FIG. 7 is schematic diagram of a male connector, according to an example.

FIG. 8 is schematic diagram of a male connector, according to an example.

FIG. 9 includes schematic diagrams of male connectors and female connectors, according to an example.

FIG. 10 is a schematic diagram of a system, according to an example.

FIG. 11 is a schematic diagram of a system, according to an example.

FIG. 12 is a schematic diagram of a hook, according to an example.

FIG. 13 is a schematic diagram of a system, according to an example.

FIG. 14 is a block diagram of a method, according to an example.

FIG. 15 is a block diagram of a method, according to an example.

FIG. 16 is a block diagram of a method, according to an example.

FIG. 17 is a block diagram of a method, according to an example.

FIG. 18 is a block diagram of a method, according to an example.

FIG. 19 is a block diagram of a method, according to an example.

FIG. 20 is a block diagram of a method, according to an example.

FIG. 21 is a block diagram of a method, according to an example.

FIG. 22 is a schematic diagram of a system, according to an example.

FIG. 23 is a schematic diagram of a system, according to an example.

FIG. 24 is a schematic diagram of a system, according to an example.

FIG. 25 is a schematic diagram of a system, according to an example.

FIG. 26 is a schematic diagram of a system, according to an example.

FIG. 27 is a schematic diagram of a system, according to an example.

FIG. 28 is a schematic diagram of a system, according to an example.

FIG. 29 is a schematic diagram of a system, according to an example.

FIG. 30 is a schematic diagram of a system, according to an example.

FIG. 31 is a schematic diagram of a system, according to an example.

FIG. 32 is a schematic diagram of a system, according to an example.

### DETAILED DESCRIPTION

This disclosure includes examples that can help alleviate some inconveniences of previous systems and methods used for peritoneal dialysis. Conventional methods and systems for peritoneal dialysis require the use and disposal of several disposable tubing lines for every dialysis cycle. Storing, connecting, disconnecting, and disposing such tubing lines can be burdensome and costly. As such, patients, providers, and payers would benefit from a system that does not require the use of as many disposable tubing lines. The system described herein eliminates the use of all disposable tubing sets, the only exception being the rare use of a single disposable tubing line on a first use for air removal. The system is configurable into a closed loop such that all internal components can be heated and disinfected between cycles.

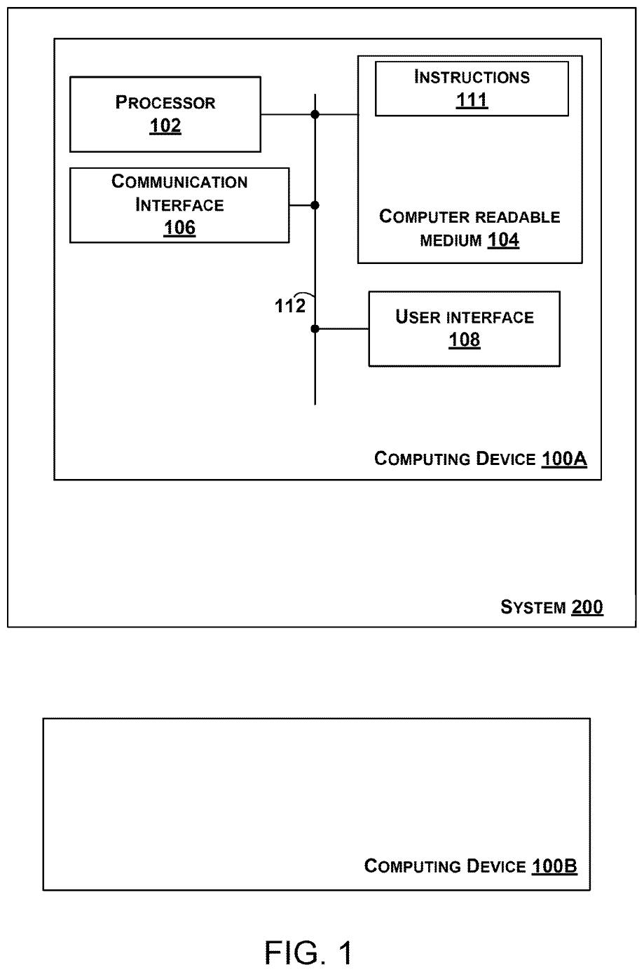

FIG. 1 is a block diagram of a system 200 for peritoneal dialysis. The system 200 includes a computing device 100A. In some examples, the computing device 100A directly controls the system 200, but in other examples, the computing device 100B can control the system 200 by sending instructions to the computing device 100A via a wired or wireless connection. For example, the computing device 100B can take the form of a tablet computer, a laptop computer, a smartphone, etc. The features and components of the computing device 100 described below can refer to the computing device 100A and/or the computing device 100B in various examples. Further features of the system 200 are detailed in subsequent Figures.

The computing device 100 includes one or more processors 102, a non-transitory computer readable medium 104, a communication interface 106, and a user interface 108. Components of the computing device 100 are linked together by a system bus, network, or other connection mechanism 112.

The one or more processors 102 can be any type of processor(s), such as a microprocessor, a field programmable gate array, a digital signal processor, a multicore processor, etc., coupled to the non-transitory computer readable medium 104.

The non-transitory computer readable medium 104 can be any type of memory, such as volatile memory like random access memory (RAM), dynamic random access memory (DRAM), static random access memory (SRAM), or non-volatile memory like read-only memory (ROM), flash memory, magnetic or optical disks, or compact-disc read-only memory (CD-ROM), among other devices used to store data or programs on a temporary or permanent basis.

Additionally, the non-transitory computer readable medium 104 can store instructions 111. The instructions 111 are executable by the one or more processors 102 to cause the computing device 100 to perform any of the functions or methods described herein.

The communication interface 106 can include hardware to enable communication within the computing device 100 and/or between the computing device 100 and one or more other devices. The hardware can include any type of input and/or output interfaces, a universal serial bus (USB), PCI Express, transmitters, receivers, and antennas, for example. The communication interface 106 can be configured to facilitate communication with one or more other devices, in accordance with one or more wired or wireless communication protocols. For example, the communication interface 106 can be configured to facilitate wireless data communication for the computing device 100 according to one or more wireless communication standards, such as one or more Institute of Electrical and Electronics Engineers (IEEE) 801.11 standards, ZigBee standards, Bluetooth standards, etc. As another example, the communication interface 106 can be configured to facilitate wired data communication with one or more other devices. The communication interface 106 can also include analog-to-digital converters (ADCs) or digital-to-analog converters (DACs) that the computing device 100 can use to control various components of the computing device 100 or external devices.

The user interface 108 can include any type of display component configured to display data. As one example, the user interface 108 can include a touchscreen display. As another example, the user interface 108 can include a flat-panel display, such as a liquid-crystal display (LCD) or a light-emitting diode (LED) display. The user interface 108 can include one or more pieces of hardware used to provide data and control signals to the computing device 100. For instance, the user interface 108 can include a mouse or a pointing device, a keyboard or a keypad, a microphone, a touchpad, or a touchscreen, among other possible types of user input devices. Generally, the user interface 108 can enable an operator to interact with a graphical user interface (GUI) provided by the computing device 100 (e.g., displayed by the user interface 108).

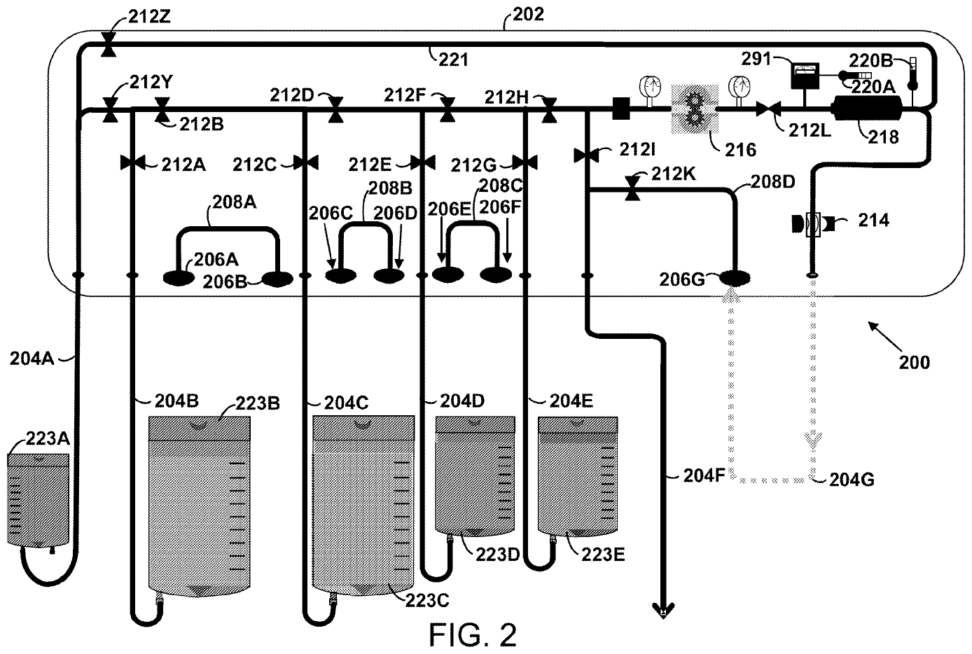

FIG. 2 is a schematic diagram of the system 200 configured for operation in a priming mode. The system 200 includes a housing 202, a water supply line 204A, a dialysate supply line 204B, a dialysate supply line 204C, a dialysate supply line 204D, a dialysate supply line 204E, a drain line 204F, and a patient line 204G each extending from the housing 202. The system 200 also includes a port 206A, a port 206B, a port 206C, a port 206D, a port 206E, and a port 206F that are accessible from outside the housing 202. The system 200 also includes a flush line 208A, a flush line 208B, a flush line 208C, and a flush line 208D each contained within the housing 202.

The flush line 208A terminates at the port 206A and at the port 206B, the flush line 208B terminates at the port 206C and the port 206D, the flush line 208C terminates at the port 208E and the port 206F, and the flush line 208D terminates at the port 206G and at the drain line 204F within the housing 202. The valve 212K is operable to open or close the connection between the flush line 208D and the drain line 204F.

The system 200 also includes a water supply bag 223A, a dialysate supply bag 223B, a dialysate supply bag 223C, a dialysate supply bag 223D, and a dialysate supply bag 223E.

The system 200 also includes a bypass line 221, a turbidity sensor 214, a heater 218, a temperature sensor 220A, a temperature sensor 220B, a conductivity sensor 291, a pump 216, a valve 212A, a valve 212B, a valve 212C, a valve 212D, a valve 212E, a valve 212F, a valve 212G, a valve 212H, a valve 212I, a valve 212J, a valve 212K, a valve 212L, a valve 212Y, and a valve 212Z.

The housing 202 is typically a metal enclosure suitable for withstanding high temperatures and protecting components inside the housing 202.

The lines 204 are generally medical grade tubing composed of ethylene propylene diene terpolymer (EPDM), silicone rubber, polypropylene, polyvinyl chloride (PVC), or polyethylene.

The ports 206 can take the form of female connectors that are described in more detail below.

The flush lines 208 are generally medical grade tubing composed of ethylene propylene diene terpolymer (EPDM), silicone rubber, polypropylene, polyvinyl chloride (PVC), polyvinylidene fluoride (PVDF), fluorinated ethylene propylene (FEP), or polyethylene.

The valves 212 are configured to receive two tubing connections and direct fluid flow accordingly.

The turbidity sensor 214 is configured to generate output representing the turbidity of fluid entering or leaving the patient and to provide the output to the computing device 100.

The pump 216 is configured to force fluid bidirectionally according to control input provided by the computing device 100.

The conductivity sensor 291 is configured to generate output representing the electrical conductivity of fluid entering or leaving the heater 218 and to provide the output to the computing device 100.

The temperature sensor 220A and the temperature sensor 220B are configured to generate outputs representing the temperature of fluid on opposite ends of the heater 218 and to provide the output to the computing device 100.

The bags 223 are plastic medical supply bags configured for containing liquid medical supplies.

In various modes of operation described herein, the port 206A is configured to connect to the dialysate supply line 204B, the port 206B is configured to connect to the dialysate supply line 204C, the port 206C is configured to connect to the water supply line 204A, the port 206D is configured to connect to the drain line 204F, the port 206E is configured to connect to the dialysate supply line 204D, the port 206F is configured to connect to the dialysate supply line 204E, and the port 206G is configured to connect to the patient line 204G.

As noted above, FIG. 2 shows the system 200 configured for operation in a priming mode.

In a first stage of the priming mode, the valves 212 are configured as follows:

| Valve | Setting |
|---|---|
| 212A | closed |
| 212B | closed |
| 212C | closed |
| 212D | closed |
| 212E | closed |
| 212F | closed |
| 212G | open |
| 212H | open |
| 212I | closed |
| 212K | open |
| 212L | open |
| 212Y | closed |
| 212Z | closed |

In the first stage of the priming mode, the dialysate supply line 204B is connected to the dialysate supply bag 223B, the dialysate supply line 204C is connected to the dialysate supply bag 223C, the dialysate supply line 204D is connected to the dialysate supply bag 223D, the dialysate supply line 204E is connected to the dialysate supply bag 223E, the drain line 204F is placed in a sink, bathtub, or toilet etc. or is connected to a drain bag, and the patient line 204G is connected to the port 206G. The pump 216 draws supply dialysate through the dialysate supply line 204E, the valve 212H, and the pump 216, and forces the supply dialysate through the valve 212L, the heater 218, the patient line 204G, the flush line 208D, the valve 212K, and the drain line 204F. The glucose concentration of the supply dialysate within the dialysate supply bag 223E is generally higher than the glucose concentration of the supply dialysate within the dialysate supply bags 223B-D.

In a second stage of the priming mode, the valves 212 are configured as follows:

| Valve | Setting |
|---|---|
| 212A | open |
| 212B | open |
| 212C | open |
| 212D | open |
| 212E | open |
| 212F | open |
| 212G | closed |
| 212H | open |
| 212I | closed |
| 212K | open |
| 212L | open |
| 212Y | closed |
| 212Z | closed |

In the second stage of the priming mode, the dialysate supply line 204B is connected to the dialysate supply bag 223B, the dialysate supply line 204C is connected to the dialysate supply bag 223C, the dialysate supply line 204D is connected to the dialysate supply bag 223D, the dialysate supply line 204E is connected to the dialysate supply bag 223E, the drain line 204F is placed in a sink, bathtub, or toilet etc. or is connected to a drain bag, and the patient line 204G is connected to the port 206G. The pump 216 draws supply dialysate through the dialysate supply lines 204B-D and forces the supply dialysate through the valve 212H, the pump 216, the valve 212L, the heater 218, the patient line 204G, the flush line 208D, the valve 212K, and the drain line 204F.

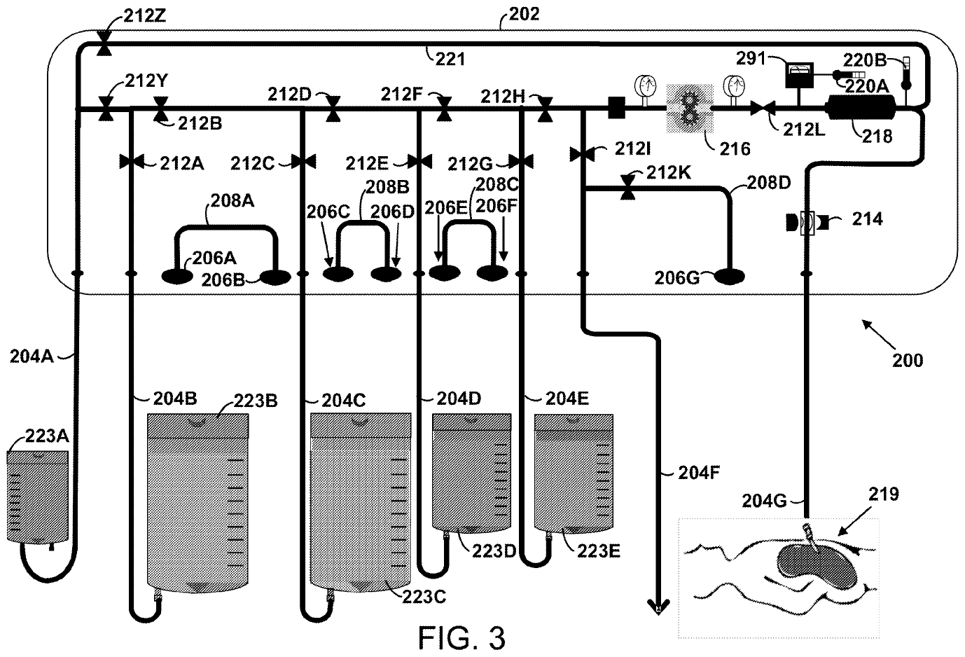

FIG. 3 is a schematic diagram of the system 200 configured for operation in a patient drain mode. In the patient drain mode, the valves 212 are configured as follows:

| Valve | Setting |
|---|---|
| 212A | closed |
| 212B | closed |
| 212C | closed |
| 212D | closed |
| 212E | closed |
| 212F | closed |
| 212G | closed |
| 212H | closed |
| 212I | open |
| 212K | closed |
| 212L | open |
| 212Y | closed |
| 212Z | closed |

In the patient drain mode, the patient line 204G is connected to the patient's peritoneum 219 via a catheter and the pump 216 draws spent dialysate through the patient line 204G, the heater 218, and the valve 212L, and forces the spent dialysate through the valve 212I and the drain line 204F.

FIG. 3 is also a schematic diagram of the system 200 configured for operation in a patient fill mode. In the patient fill mode, the valves 212 are configured as follows:

| Valve | Setting |
|---|---|
| 212A | open |
| 212B | open |
| 212C | open |
| 212D | open |
| 212E | open |
| 212F | open |
| 212G | closed |
| 212H | open |
| 212I | closed |
| 212K | closed |
| 212L | open |
| 212Y | closed |
| 212Z | closed |

In the patient mode, the dialysate supply line 204B is connected to the dialysate supply bag 223B, the dialysate supply line 204C is connected to the dialysate supply bag 223C, the dialysate supply line 204D is connected to the dialysate supply bag 223D, the dialysate supply line 204E is connected to the dialysate supply bag 223E, and the patient line 204G is connected to the patient's peritoneum 219 via a catheter. The pump 216 draws supply dialysate through the dialysate supply lines 204B-D and forces the supply dialysate through the valve 212L, the heater 218, and the patient line 204G and into the patient's peritoneum 219. During the patient fill mode, the heater 218 heats the supply dialysate to a temperature of approximately 98.6 degrees Fahrenheit.

In a final patient fill mode, the valves 212 are configured as follows:

| Valve | Setting |
|---|---|
| 212A | closed |
| 212B | closed |
| 212C | closed |
| 212D | closed |
| 212E | closed |
| 212F | closed |
| 212G | open |
| 212H | open |
| 212I | closed |
| 212K | closed |
| 212L | open |
| 212Y | closed |
| 212Z | closed |

In the final patient fill mode, the dialysate supply line 204B is connected to the dialysate supply bag 223B, the dialysate supply line 204C is connected to the dialysate supply bag 223C, the dialysate supply line 204D is connected to the dialysate supply bag 223D, the dialysate supply line 204E is connected to the dialysate supply bag 223E, and the patient line 204G is connected to the patient's peritoneum 219 via a catheter. The pump 216 draws supply dialysate through the dialysate supply line 204E and forces the supply dialysate through the valve 212L, the heater 218, and the patient line 204G and into the patient's peritoneum 219. During the final patient fill mode, the heater 218 heats the supply dialysate to a temperature of approximately 98.6 degrees Fahrenheit.

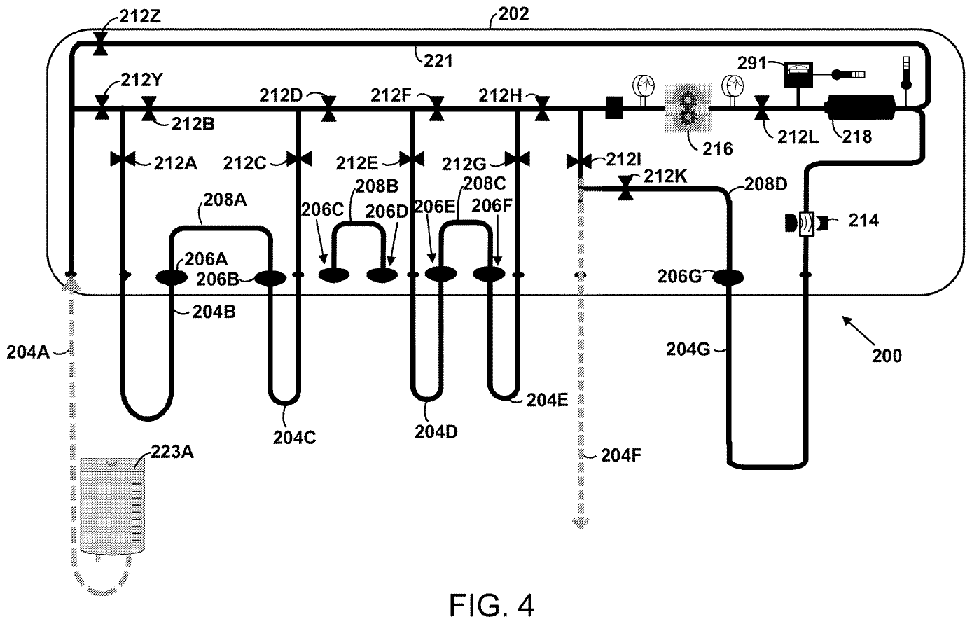

FIG. 4 is a schematic diagram of the system 200 configured for operation in a flush mode. In a first stage of the flush mode, the valves 212 are configured as follows:

| Valve | Setting |
|---|---|
| 212A | closed |
| 212B | open |
| 212C | closed |
| 212D | open |
| 212E | closed |
| 212F | open |
| 212G | closed |
| 212H | open |
| 212I | closed |
| 212K | open |
| 212L | open |
| 212Y | open |
| 212Z | closed |

In the first stage of the flush mode, the water supply line 204A is connected to the water supply bag 223A, the dialysate supply line 204B is connected to the port 206A, the dialysate supply line 204C is connected to the port 206B, the dialysate supply line 204D is connected to the port 206E, the dialysate supply line 204E is connected to the port 206F, the drain line 204F is placed in a sink or connected to a drain bag, and the patient line 204G is connected to the port 206G. The pump 216 draws water through the water supply line 204A, the valve 212Y, the valve 212B, the valve 212D, the valve 212F, and the valve 212H and forces the water through the valve 212L, the heater 218, the patient line 204G, the flush line 208D, the valve 212K, and the drain line 204F.

In a second stage of the flush mode, the valves 212 are configured as follows:

| Valve | Setting |
|---|---|
| 212A | open |
| 212B | closed |
| 212C | open |
| 212D | open |
| 212E | open |
| 212F | closed |
| 212G | open |
| 212H | open |
| 212I | closed |
| 212K | open |
| 212L | open |
| 212Y | open |
| 212Z | closed |

In the second stage of the flush mode, the water supply line 204A is connected to the water supply bag 223A, the dialysate supply line 204B is connected to the port 206A, the dialysate supply line 204C is connected to the port 206B, the dialysate supply line 204D is connected to the port 206E, the dialysate supply line 204E is connected to the port 206F, the drain line 204F is placed in a sink or connected to a drain bag, and the patient line 204G is connected to the port 206G. The pump 216 draws water through the water supply line 204A, the valve 212Y, the valve 212A, the dialysate supply line 204B, the flush line 208A, the dialysate supply line 204C, the valve 212C, the valve 212D, the valve 212E, the dialysate supply line 204D, the flush line 208C, the dialysate supply line 204E, the valve 212G, the valve 212H, and forces the water through the valve 212L, the heater 218, the patient line 204G, the flush line 208D, the valve 212K, and the drain line 204F.

In a third stage of the flush mode, the valves 212 are configured as follows:

| Valve | Setting |
|---|---|
| 212A | closed |
| 212B | closed |
| 212C | closed |
| 212D | closed |
| 212E | closed |
| 212F | closed |
| 212G | closed |
| 212H | closed |
| 212I | open |
| 212K | closed |
| 212L | open |
| 212Y | closed |
| 212Z | open |

In the third stage of the flush mode, the water supply line 204A is connected to the water supply bag 223A, the dialysate supply line 204B is connected to the port 206A, the dialysate supply line 204C is connected to the port 206B, the dialysate supply line 204D is connected to the port 206E, the dialysate supply line 204E is connected to the port 206F, the drain line 204F is placed in a sink or connected to a drain bag, and the patient line 204G is connected to the port 206G. The pump 216 draws water through the water supply line 204A, through the valve 212Z, the bypass line 221, the heater 218, and the valve 212L, and forces the water through the valve 212I and the drain line 204F.

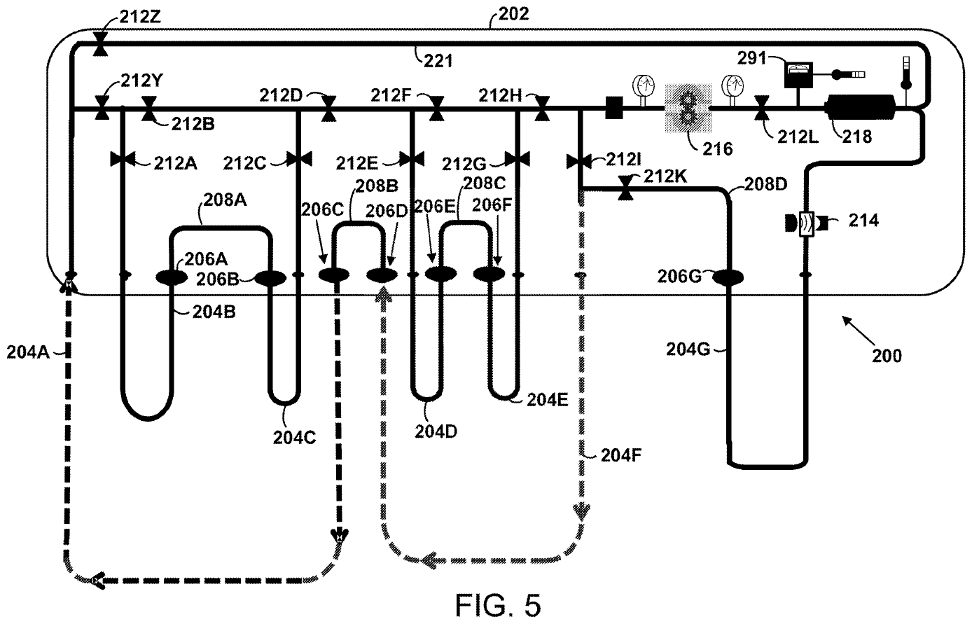

FIG. 5 is a schematic diagram of the system 200 configured for operation in a disinfect mode. In a first stage of a disinfect mode, the valves 212 are configured as follows:

| Valve | Setting |
|---|---|
| 212A | closed |
| 212B | open |
| 212C | closed |
| 212D | open |
| 212E | closed |
| 212F | open |
| 212G | closed |
| 212H | open |
| 212I | closed |
| 212K | open |
| 212L | open |
| 212Y | open |
| 212Z | closed |

In the first stage of the disinfect mode, the water supply line 204A is connected to the port 206C, the dialysate supply line 204B is connected to the port 206A, the dialysate supply line 204C is connected to the port 206B, the dialysate supply line 204D is connected to the port 206E, the dialysate supply line 204E is connected to the port 206F, the drain line 204F is connected to the port 206D, and the patient line 204G is connected to the port 206G.

In the first stage of the disinfect mode, the pump 216 circulates water through the water supply line 204A, the valve 212Y, the valve 212B, the valve 212D, the valve 212F, the valve 212H, the pump 216, the valve 212L, the heater 218, the patient line 204G, the flush line 208D, the valve 212K, the drain line 204F, the flush line 208B, and back to the water supply line 204A. The heater 218 heats the water to about 80 degrees Celsius.

In a second stage of the disinfect mode, the valves 212 are configured as follows:

| Valve | Setting |
|---|---|
| 212A | open |
| 212B | closed |
| 212C | open |
| 212D | open |
| 212E | open |
| 212F | closed |
| 212G | open |
| 212H | open |
| 212I | closed |
| 212K | open |
| 212L | open |
| 212Y | open |
| 212Z | closed |

In the second stage of the disinfect mode, the pump 216 circulates water through the water supply line 204A, the valve 212Y, the valve 212A, the dialysate supply line 204B, the flush line 208A, the dialysate supply line 204C, the valve 212C, the valve 212D, the valve 212E, the dialysate supply line 204D, the flush line 208C, the dialysate supply line 204E, the valve 212G, the valve 212H, the pump 216, the valve 212L, the heater 218, the patient line 204G, the flush line 208D, the valve 212K, the drain line 204F, the flush line 208B, and back to the water supply line 204A. The heater 218 heats the water to about 80 degrees Celsius.

In a third stage of the disinfect mode, the valves 212 are configured as follows:

| Valve | Setting |
|---|---|
| 212A | closed |
| 212B | closed |
| 212C | closed |
| 212D | closed |
| 212E | closed |
| 212F | closed |
| 212G | closed |
| 212H | closed |
| 212I | open |
| 212K | closed |
| 212L | open |
| 212Y | closed |
| 212Z | open |

In the third stage of the disinfect mode, the pump 216 circulates water through the water supply line 204A, the valve 212Z, the bypass line 221, the heater 218, the valve 212L, the pump 216, the valve 212I, the drain line 204F, the flush line 208, and back to the water supply line 204A. The heater 218 heats the water to about 80 degrees Celsius.

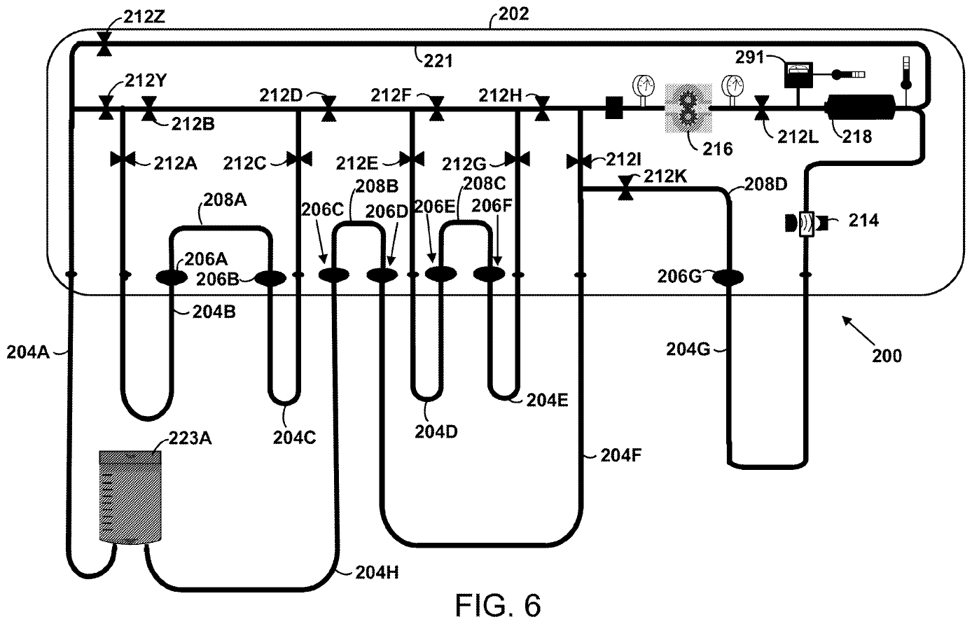

FIG. 6 is a schematic diagram of the system 200 configured for operation in an air removal mode. The system 200 includes a disposable priming line 204H which is used in the air removal mode. In a first stage of the air removal mode, the valves 212 are configured as follows:

| Valve | Setting |
|---|---|
| 212A | closed |
| 212B | open |
| 212C | closed |
| 212D | open |
| 212E | closed |
| 212F | open |
| 212G | closed |
| 212H | open |
| 212I | closed |
| 212K | open |
| 212L | open |
| 212Y | open |
| 212Z | closed |

In the first stage of the air removal mode, the water supply line 204A is connected to a first bag port of the water supply bag 223A, the dialysate supply line 204B is connected to the port 206A, the dialysate supply line 204C is connected to the port 206B, the dialysate supply line 204D is connected to the port 206E, the dialysate supply line 204E is connected to the port 206F, the disposable priming line 204H is connected to the port 206C and to a second bag port of the water supply bag 223A, the drain line 204F is connected to the port 206G, and the patient line 204G is connected to the port 206D.

In the first stage of the air removal mode, the pump 216 circulates water through the water supply line 204A, the valve 212Y, the valve 212B, the valve 212D, the valve 212F, the valve 212H, the pump 216, the valve 212L, the heater 218, the patient line 204G, the flush line 208D, the valve 212K, the drain line 204F, the flush line 208B, and the disposable priming line 204H, thereby trapping air received from the fluid paths and the disposable priming line 204H in the water supply bag 223A.

In a second stage of the air removal mode, the valves 212 are configured as follows:

| Valve | Setting |
|---|---|
| 212A | open |
| 212B | closed |
| 212C | open |
| 212D | open |
| 212E | open |
| 212F | closed |
| 212G | open |
| 212H | open |
| 212I | closed |
| 212K | open |
| 212L | open |
| 212Y | open |
| 212Z | closed |

In the second stage of the air removal mode, the pump 216 circulates water through the water supply line 204A, the valve 212Y, the valve 212A, the valve 212C, the valve 212D, the valve 212E, the valve 212G, the valve 212H, the pump 216, the valve 212L, the heater 218, the patient line 204G, the flush line 208D, the valve 212K, the drain line 204F, the flush line 208B, and the disposable priming line 204H, thereby trapping air received from the fluid paths and the disposable priming line 204H in the water supply bag 223A.

In a third stage of the air removal mode the valves 212 are configured as follows:

| Valve | Setting |
|---|---|
| 212A | closed |
| 212B | closed |
| 212C | closed |
| 212D | closed |
| 212E | closed |
| 212F | closed |
| 212G | closed |
| 212H | closed |
| 212I | open |
| 212K | closed |
| 212L | open |
| 212Y | closed |
| 212Z | open |

In the third stage of the air removal mode, the pump 216 circulates water through the water supply line 204A, the valve 212Z, the bypass line 221, the heater 218, the valve 212L, the pump 216, the valve 212I, the drain line 204F, the flush line 208B, and the disposable priming line 204H, thereby trapping air received from the disposable priming line 204H in the water supply bag 223A.

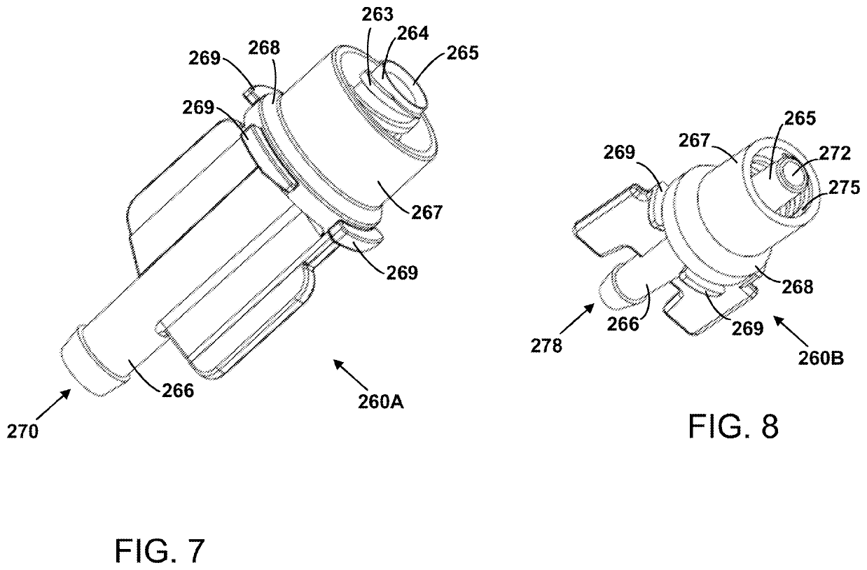

FIG. 7 is a schematic diagram of a male connector 260A. The male connector 260A includes a stem 265 having male threads 263 on an exterior surface 264 of the stem 265. An interior surface of the stem 265 defines a tube 266 that is securely connected to and in fluid communication with the water supply line 204A, one of the dialysate supply lines 204B-E, or the drain line 204F, as applicable. The male threads 263 are configured to mate with female threads of an adapter that is connected to one of the dialysate supply bags 223B-E or the water supply bag 222A.

The male connector 260A also includes a shroud 267 that surrounds a proximal portion of the stem 265 such that a distal portion of the stem 265 extends distally beyond the shroud 267. The male connector 260A also includes a circumferential sealing surface 268 proximal to the shroud 267 and lugs 269 proximal to the circumferential sealing surface 268. As shown, the exterior surface 264 is distally tapered. The male connector 260A is configured for connection to the water supply line 204A or one of the dialysate supply lines 204B-E at a proximal end 270 of the male connector 260A.

FIG. 8 is a schematic diagram of a male connector 260B. The male connector 260B includes a stem 265 having an interior surface 272 that defines a tube 266 that is securely connected to and in fluid communication with the patient line 204G. The male connector 260B also includes a shroud 267 that surrounds a proximal portion of the stem 265 such that a distal portion of the stem 265 extends distally beyond the shroud 267. The shroud 267 includes male threads 275 on an interior surface of the shroud 267 that are configured to mate with female threads of an adapter that is connected to a catheter that is inserted into the patient's peritoneum. The male connector 260B also includes a circumferential sealing surface 268 proximal to the shroud 267 and lugs 269 that are proximal to the circumferential sealing surface 268. The exterior surface of the stem 265 is distally tapered. The male connector 260B is configured for connection to the patient line 204G at a proximal end 278 of the male connector 260B.

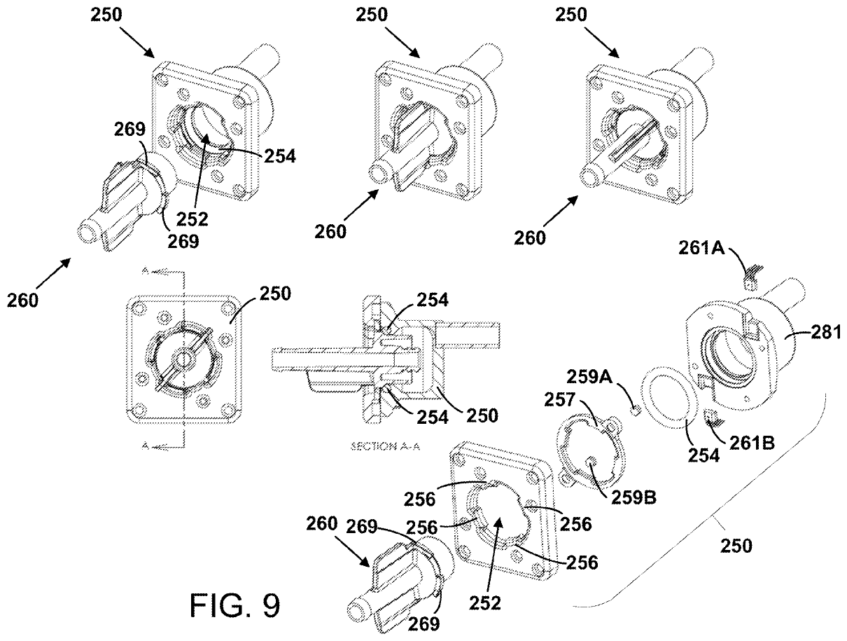

FIG. 9 is a collection of schematic diagrams of a female connector 250 and a male connector 260 that can take the form of the male connector 260A or the male connector 260B. The lines 204 and the ports 206 shown in FIGS. 2-6 can connect to each other in various ways. For example, the ports 206 can each include (e.g., terminate at) a female connector 250 that is sized to accommodate a particular male connector 260 that terminates the corresponding line 204.

The female connector 250 includes an opening 252, an O-ring 254 surrounding the opening 252, and lugs 256 disposed around the opening 252. The male connector 260 is configured to be connected to the female connector 250 by inserting the male connector 260 axially into the opening 252 such that the lugs 269 move axially past the lugs 256, and rotating the male connector 260 such that the lugs 269 move behind the lugs 256. This seals the circumferential sealing surface 268 against the O-ring 254 and exposes the shroud 267 and the stem 265 to one of the flush lines 208A-D. Thus, the shroud 267 and the stem 265 are exposed to cleansing hot water during the disinfect cycle.

The female connector 250 includes a sensing ring 257 that further includes a permanent magnet 259A and a permanent magnet 259B. The sensing ring 257 and the permanent magnets 259 are configured to rotate as the male connector 260 is rotated within the opening 252. The female connector 250 further includes a base 281 that includes a Hall effect sensor 261A and a Hall effect sensor 261B. The Hall effect sensor 261A and the Hall effect sensor 261B are configured to indicate whether the male connector 260 is properly connected to the female connector 250 based on rotational positions of the permanent magnet 259A and the permanent magnet 259B relative to the Hall effect sensor 261A and the Hall effect sensor 261B.

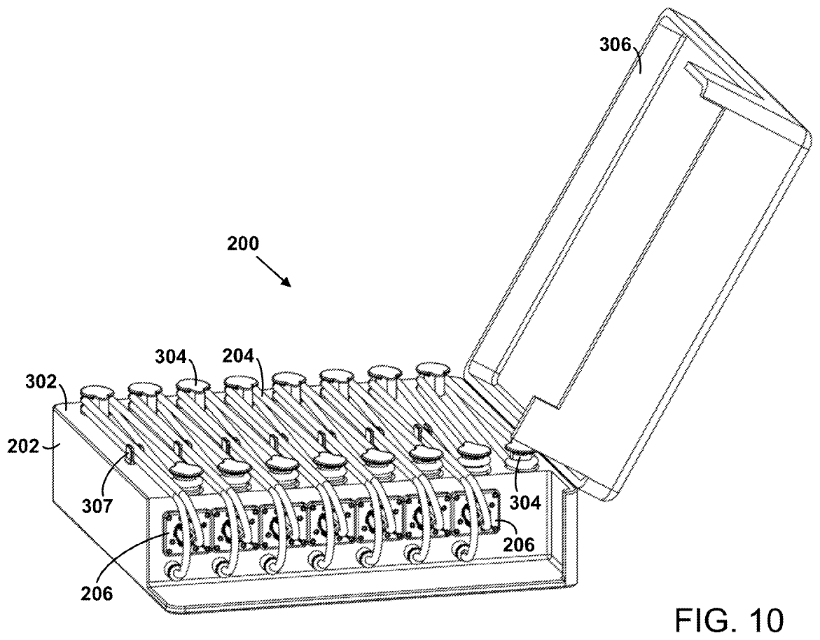

FIG. 10 is a schematic diagram of the system 200. The system 200 includes the housing 202 having an exterior storage surface 302. Typically, the housing 202 is made of metal and is internally insulated to reduce heat escaping from the housing 202 during the disinfect mode. The system 200 further includes hooks 304 disposed on the exterior storage surface 302. The water supply line 204A, the dialysate supply lines 204B-E, the drain line 204F, and the patient line 204G are configured to be wrapped around the hooks 304 for storage during the disinfect mode of the system 200.

The system 200 also includes a lid 306 that is rotatable (e.g., about a hinge) to be in an open or a closed position. FIG. 10 shows the open position in which the lines 204 and the ports 206 are accessible. In the closed position, the lines 204 and the ports 206 are not accessible. Thus, the lid 306 covers the exterior storage surface 302, the water supply line 204A, the dialysate supply lines 204B-E, the drain line 204F, and the patient line 204G during the disinfect mode. The system 200 is configured to lock the lid 306 such that the lid 306 cannot be opened during the disinfect mode.

As shown, the system 200 also includes clips 307 positioned between the hooks 304. Each of the clips 307 is configured to hold the water supply line 204A, one of the dialysate supply lines 204B-E, the drain line 204F, or the patient line 204G between the plurality of hooks 304 during the disinfect mode.

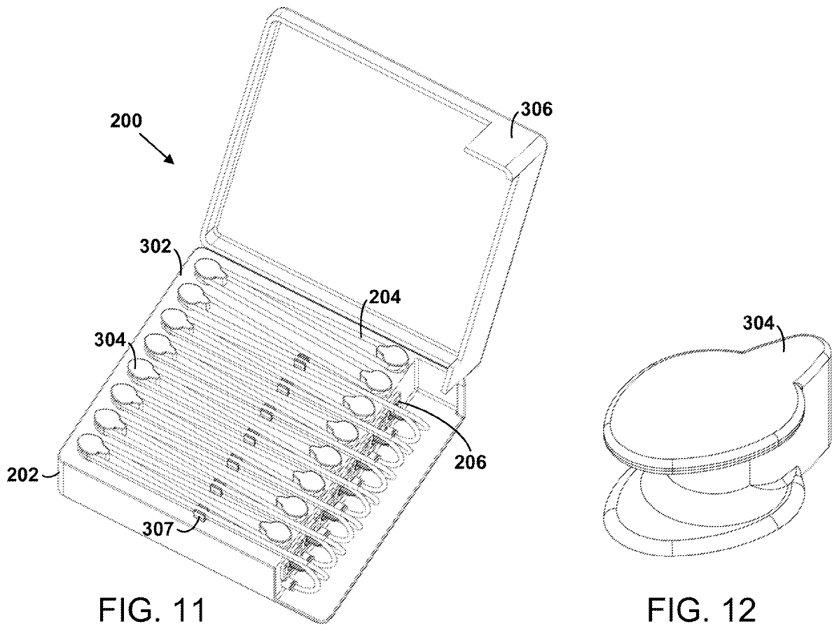

FIG. 11 is a schematic diagram of the system 200 as an overhead view.

FIG. 12 is a close up view of a hook 304.

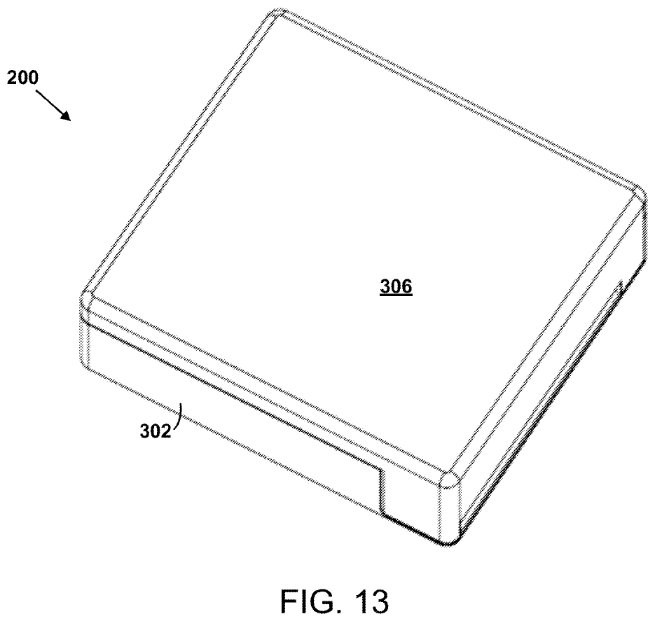

FIG. 13 is a schematic diagram of the lid 306 in the closed position.

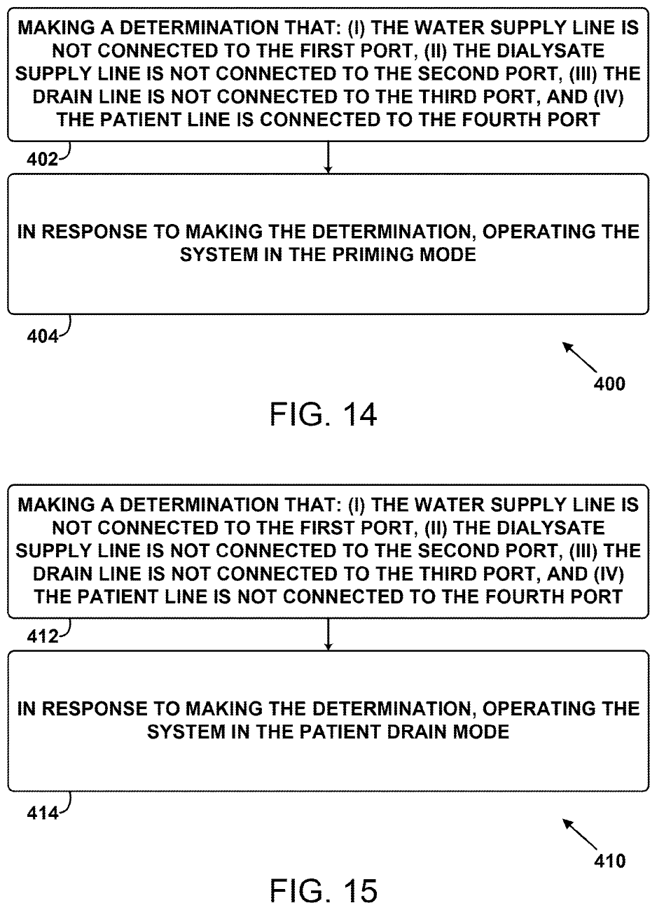

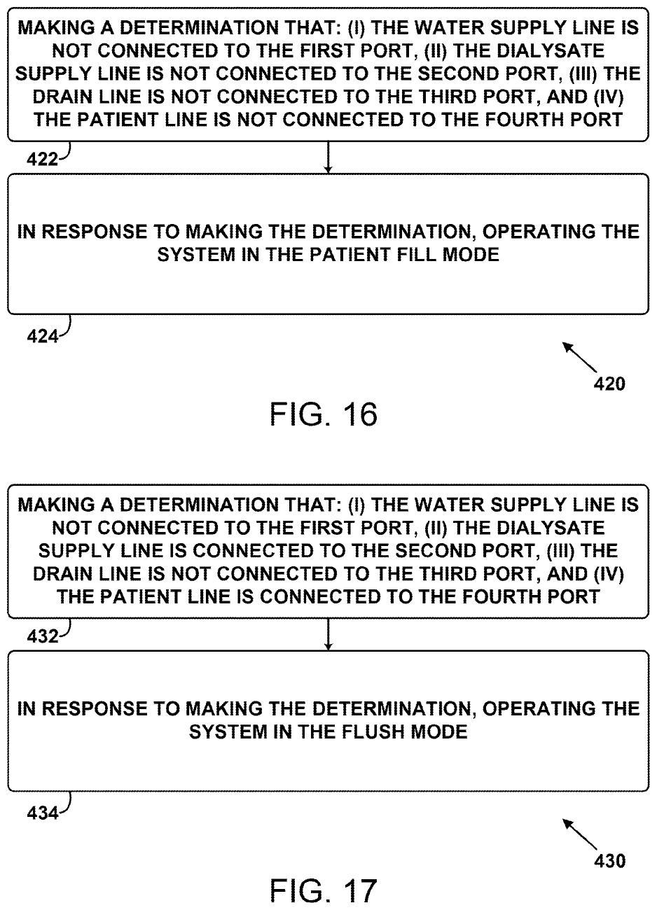

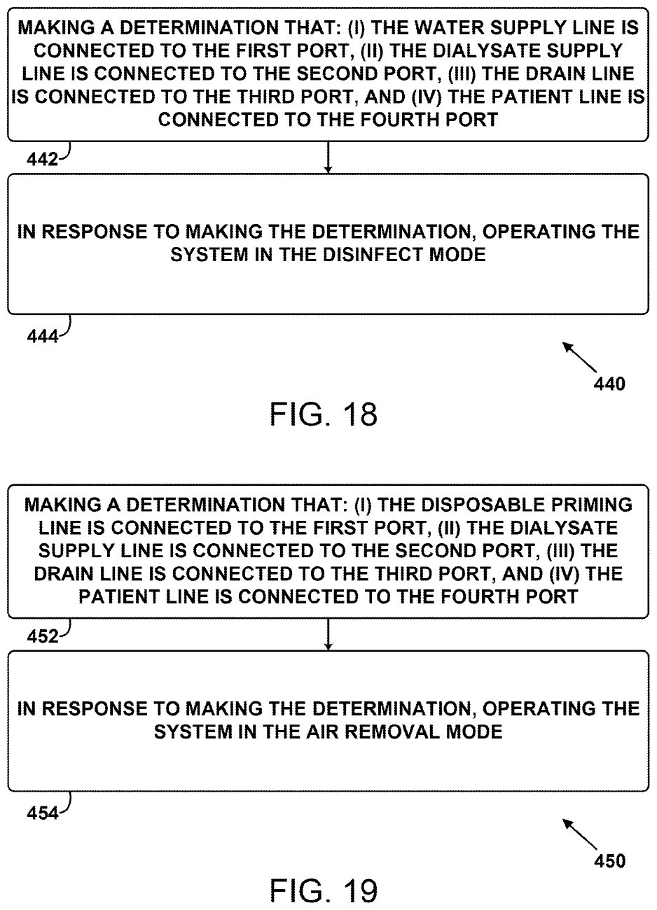

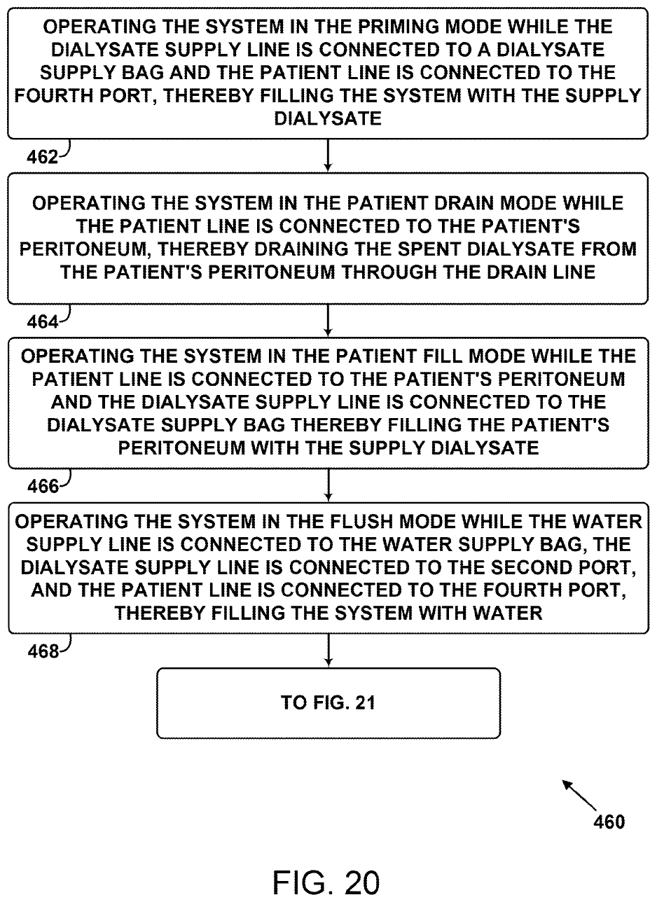

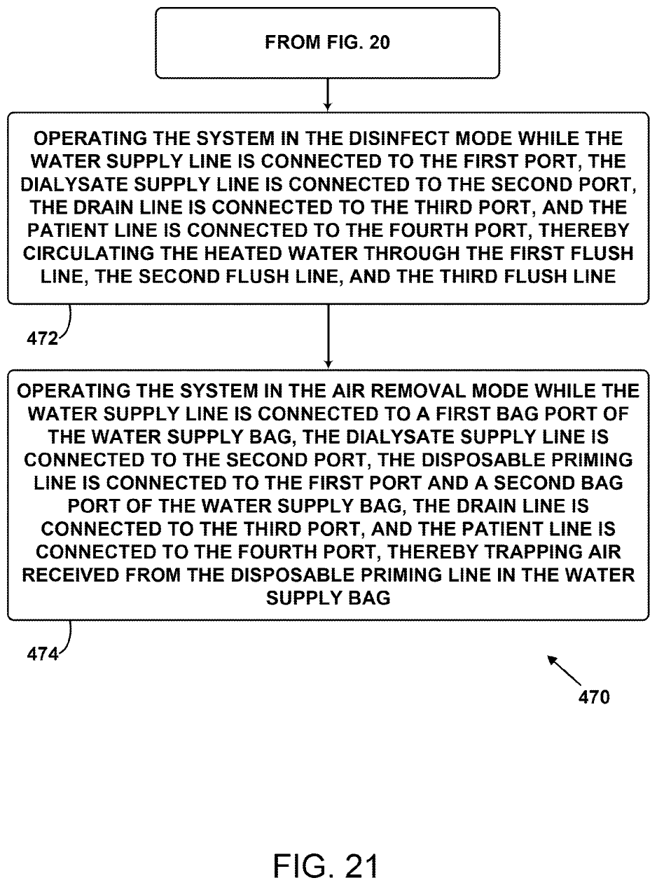

FIGS. 14-21 are block diagrams of a method 400, a method 410, a method 420, a method 430, a method 440, a method 450, a method 460, and a method 470, which in some examples are performed by the system 200 and/or manually. As shown in FIGS. 14-21, the methods 400-470 include one or more operations, functions, or actions as illustrated by blocks 402, 404, 412, 414, 422, 424, 432, 434, 442, 444, 452, 454, 462, 464, 466, 468, 472, and 474. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

At block 402, the method 400 includes the computing device 100 making a determination that: (i) the water supply line 204A is not connected to the port 206C, (ii) the dialysate supply lines 204B-204E are not connected to the ports 206A-B and 206E-F, (iii) the drain line 204F is not connected to the port 206D, and (iv) the patient line 204G is connected to the port 206G. Functionality related to block 402 is described above with reference to FIG. 2.

At block 404, the method 400 includes in response to making the determination, the computing device 100 operating the system 200 in the priming mode. Functionality related to block 404 is described above with reference to FIG. 2.

At block 412, the method 410 includes the computing device 100 making a determination that: (i) the water supply line 204A is not connected to the port 206C, (ii) the dialysate supply lines 204B-204E are not connected to the ports 206A-B and 206E-F, (iii) the drain line 204F is not connected to the port 206D, and (iv) the patient line 204G is not connected to the port 206D. Functionality related to block 412 is described above with reference to FIG. 3.

At block 414, the method 410 includes in response to making the determination, the computing device 100 operating the system 200 in the patient drain mode. Functionality related to block 414 is described above with reference to FIG. 3.

At block 422, the method 420 includes the computing device 100 making a determination that: (i) the water supply line 204A is not connected to the port 206C, (ii) the dialysate supply lines 204B-204E are not connected to the ports 206A-B and 206E-F, (iii) the drain line 204F is not connected to the port 206D, and (iv) the patient line 204G is not connected to the port 206G. Functionality related to block 422 is described above with reference to FIG. 3.

At block 424, the method 420 includes in response to making the determination, the computing device 100 operating the system 200 in the patient fill mode. Functionality related to block 424 is described above with reference to FIG. 3.

At block 432, the method 430 includes the computing device 100 making a determination that: (i) the water supply line 204A is not connected to the port 206C, (ii) the dialysate supply lines 204B-204E are connected to the ports 206A-B and 206E-F, (iii) the drain line 204F is not connected to the port 206D, and (iv) the patient line 204G is connected to the port 206G. Functionality related to block 432 is described above with reference to FIG. 4.

At block 434, the method 430 includes in response to making the determination, the computing device 100 operating the system 200 in the flush mode. Functionality related to block 434 is described above with reference to FIG. 4.

At block 442, the method 440 includes the computing device 100 making a determination that: (i) the water supply line 204A is connected to the port 206C, (ii) the dialysate supply lines 204B-204E are connected to the ports 206A-B and 206E-F, (iii) the drain line 204F is connected to the port 206D, and (iv) the patient line 204G is connected to the port 206G. Functionality related to block 442 is described above with reference to FIG. 5.

At block 444, the method 440 includes in response to making the determination, the computing device 100 operating the system 200 in the disinfect mode. Functionality related to block 444 is described above with reference to FIG. 5.

At block 452, the method 450 includes the computing device 100 making a determination that: (i) the disposable priming line 204H is connected to the port 206C, (ii) the dialysate supply lines 204B-204E are connected to the ports 206A-B and 206E-F, (iii) the drain line 204F is connected to the port 206D, and (iv) the patient line 204G is connected to the port 206G. Functionality related to block 452 is described above with reference to FIG. 6.

At block 454, the method 450 includes in response to making the determination, the computing device 100 operating the system 200 in the air removal mode. Functionality related to block 454 is described above with reference to FIG. 6.

At block 462, the method 460 includes operating the system 200 in the priming mode while the dialysate supply lines 204B-204E are connected to the dialysate supply bags 223B-E and the patient line 204G is connected to the port 206G, thereby filling the system 200 with the supply dialysate. Functionality related to block 462 is described above with reference to FIG. 2.

At block 464, the method 460 includes operating the system 200 in the patient drain mode while the patient line 204G is connected to the patient's peritoneum 219, thereby draining the spent dialysate from the patient's peritoneum 219 through the drain line 204F. Functionality related to block 464 is described above with reference to FIG. 3.

At block 466, the method 460 includes operating the system 200 in the patient fill mode while the patient line 204G is connected to the patient's peritoneum 219 and the dialysate supply lines 204B-204E are connected to the dialysate supply bags 223B-E, thereby filling the patient's peritoneum 219 with the supply dialysate. Functionality related to block 466 is described above with reference to FIG. 3.

At block 468, the method 460 includes operating the system 200 in the flush mode while the water supply line 204A is connected to the water supply bag 223A, the dialysate supply lines 204B-204E are connected to the ports 206A-B and 206E-F, and the patient line 204G is connected to the port 206G, thereby filling the system 200 with water. Functionality related to block 468 is described above with reference to FIG. 4.

At block 472, the method 470 includes operating the system 200 in the disinfect mode while the water supply line 204A is connected to the port 206C, the dialysate supply lines 204B-204E are connected to the ports 206A-B and 206E-F, the drain line 204F is connected to the port 206D, and the patient line 204G is connected to the port 206G, thereby circulating the heated water through the flush lines 208A-D. Functionality related to block 472 is described above with reference to FIG. 5.

At block 474, the method 470 includes operating the system 200 in the air removal mode while the water supply line 204A is connected to a first bag port of the water supply bag 223A, the dialysate supply lines 204B-E are connected to the ports 206A-B and 206E-F, the disposable priming line 204H is connected to the port 206C and a second bag port of the water supply bag 223A, the drain line 204F is connected to the port 206D, and the patient line 204G is connected to the port 206G, thereby trapping air received from the disposable priming line 204H in the water supply bag 223A.

### FURTHER EXAMPLE EMBODIMENTS

The present disclosure relates to the provision of peritoneal dialysis as a treatment for kidney failure using automated instruments generically known as cyclers. Typically in peritoneal dialysis, a sterile dialysate contained in a plastic bag is infused into the peritoneal cavity via an indwelling catheter. This can be accomplished manually by gravity or with the use of a cycler. The dialysate is allowed to dwell in the peritoneal cavity for a sufficient length of time (e.g., 4 hours) to yield a net removal of toxins and water after which the dialysate is drained and replaced with fresh dialysate.

Cyclers automate this process. Many current peritoneal cyclers control this process by interfacing with a sterile disposable tubing set that is connected to multiple supply bags of dialysate on the afferent end, and a drain line which is connected to a drain bag or an open drain on the efferent end. In the middle is typically a cassette of some sort which is captured in a door of the cycler. This cassette provides the means for pumping the dialysate to and from the patient, valving to close or open certain tubing lines at the appropriate times, and means for measuring or calculating the volume of fluid transported to and from the patient so that the net amount of water removed from the patient (ultrafiltration) can be calculated. Also, most often included in the tubing set is a plastic bag which is initially empty but in use, is placed on a platen on top of the cycler containing a heater which, when the bag is filled with the initial fill volume of dialysate, heats the solution to body temperature for the comfort of the patient.

Current cycler designs generally require a multitude of electromechanical components and sensors and, as with any instrument, the more components, the higher the cost, the greater the weight, the bigger the size and the lower the reliability; all of which negatively affect marketability. The same applies to the disposable tubing set; the more tubes, connectors, cassettes and bags, the more cost, the less reliable, the more time consuming they are to install and deinstall on the instrument, and the more space they consume in the residence and in landfills once discarded. Also, the current method of heating the dialysate by placing a plastic bag on a heater platen is very inefficient since much of the heat is lost to the atmosphere and the heat must be transferred from the platen through the plastic and into the liquid.

A benefit of the disclosure is the design of a cycler which: dramatically simplifies the instrument thereby reducing the size, weight, cost and operating complexity all while improving reliability, eliminates the disposable tubing set and heater bag completely thereby further reducing the cost per treatment, improving the efficiency of heating and lowering energy consumption, reducing the setup time and complexity, and reducing the number of boxes patients have to store in their home, implements a novel and very cost-effective method of measuring the volume of dialysate instilled and removed from the patient and, therefore, the ultrafiltration.

This is accomplished by: making the tubing set (fluid pathway) semi-permanent and mostly internal to the instrument rather than a daily disposable, and, designing the instrument to rinse and disinfect the fluid path automatically between treatments so that largely all the patient has to do to initiate a treatment is connect new supply bags of dialysate and their catheter to the instrument, and connect the drain line to a drain and press “GO”.

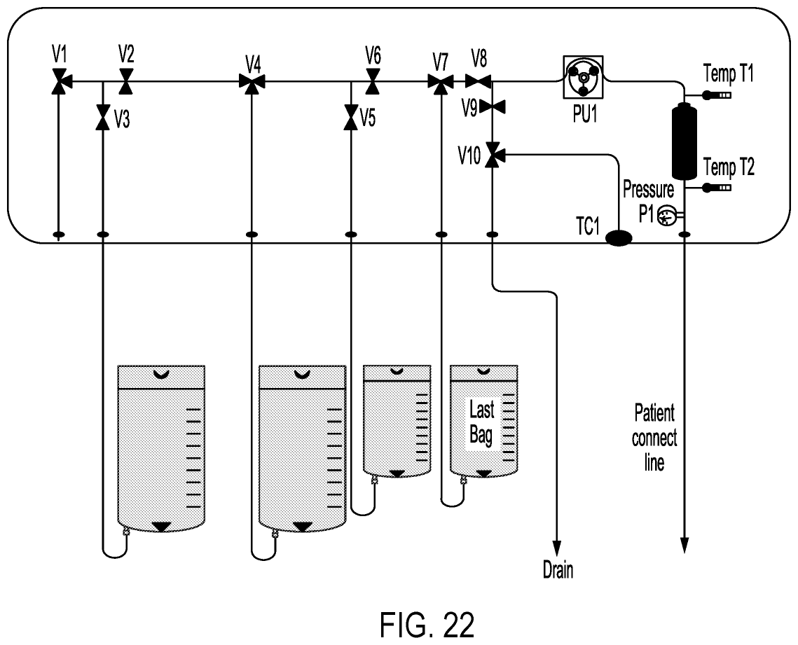

FIG. 22 discloses a peritoneal dialysis cycler with reusable fluid pathways.

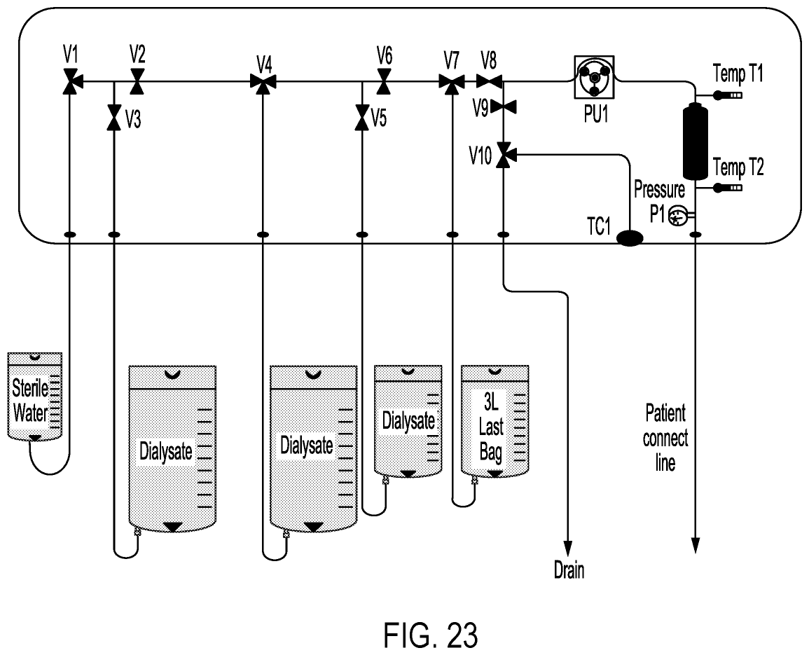

FIG. 23 discloses the cycler of FIG. 22 with attached bag of sterile water to be used in the flushing and disinfection processes.

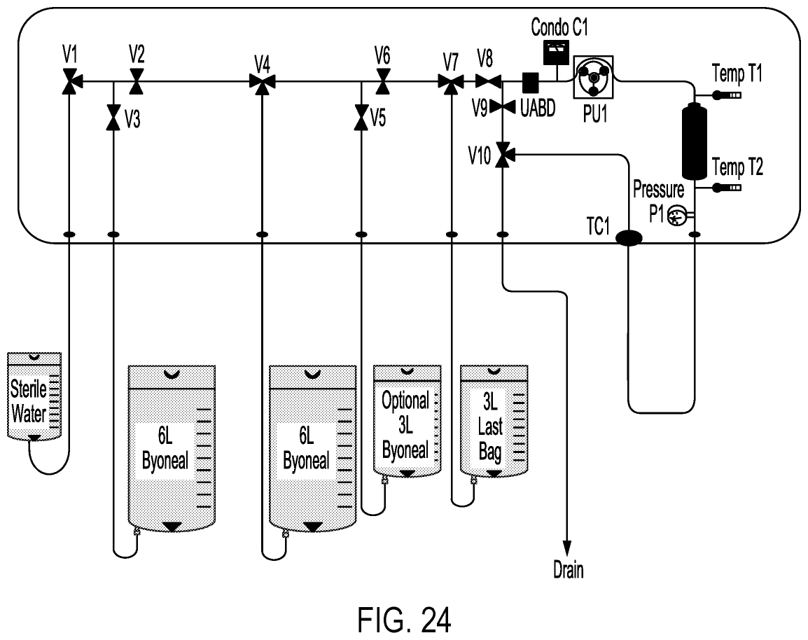

FIG. 24 discloses how the patient connection line is coupled into the machine to form a fluidic pathway to the drain line.

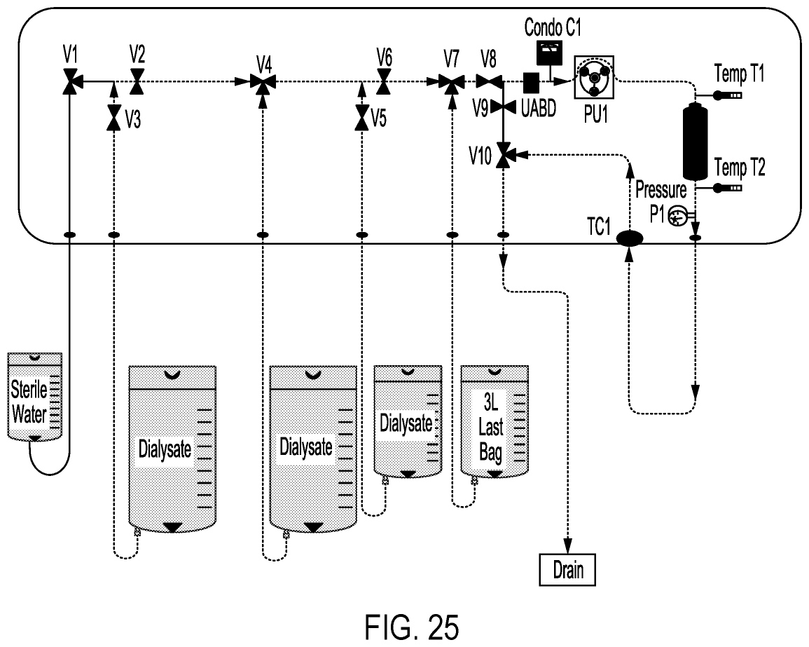

FIG. 25 discloses a graphic depiction of how the fluid pathways would be primed with dialysate.

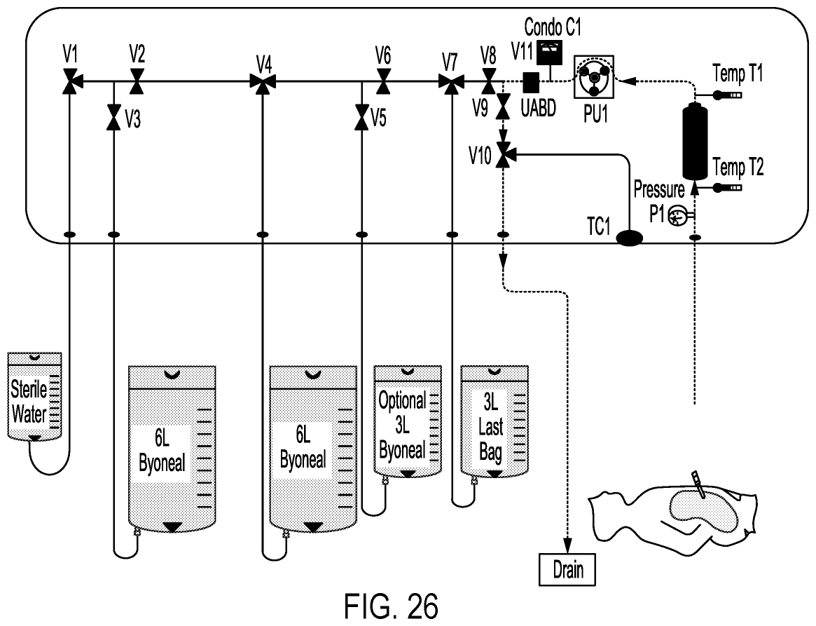

FIG. 26 discloses a graphic depiction of how the initial drain of the dialysate from the patient would prime the remaining fluid pathway segment between values V9 and V10.

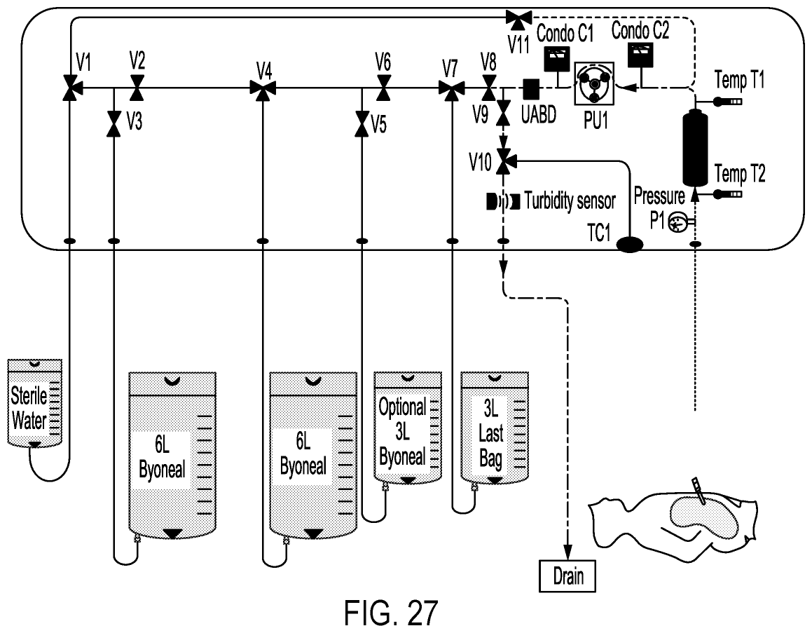

FIG. 27 discloses how pulses of water would allow the calculation of flow rate using the conductivity time-of-flight principle in the drain mode.

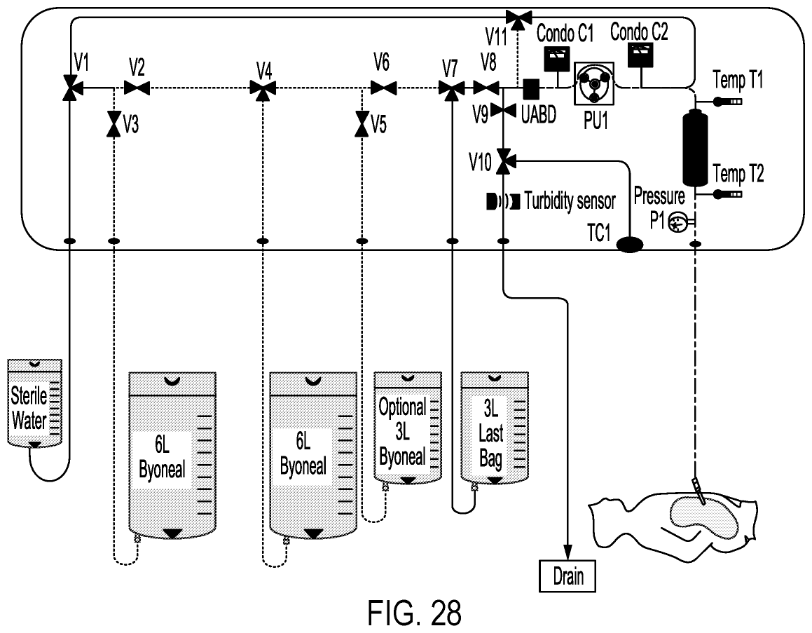

FIG. 28 discloses how pulses of water would allow the calculation of flow rate using the conductivity time-of-flight principle in the fill mode.

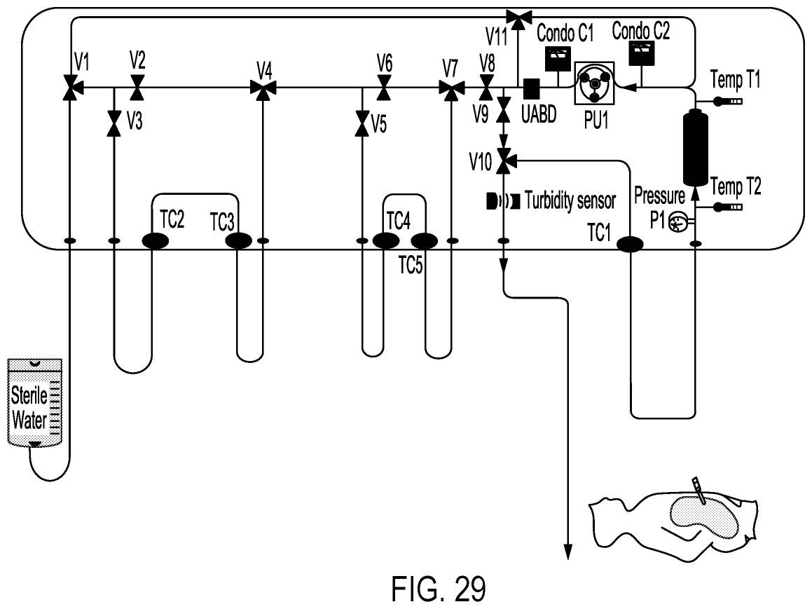

FIG. 29 discloses how the dialysate supply lines would be connected into the instrument to form flow-through circuits.

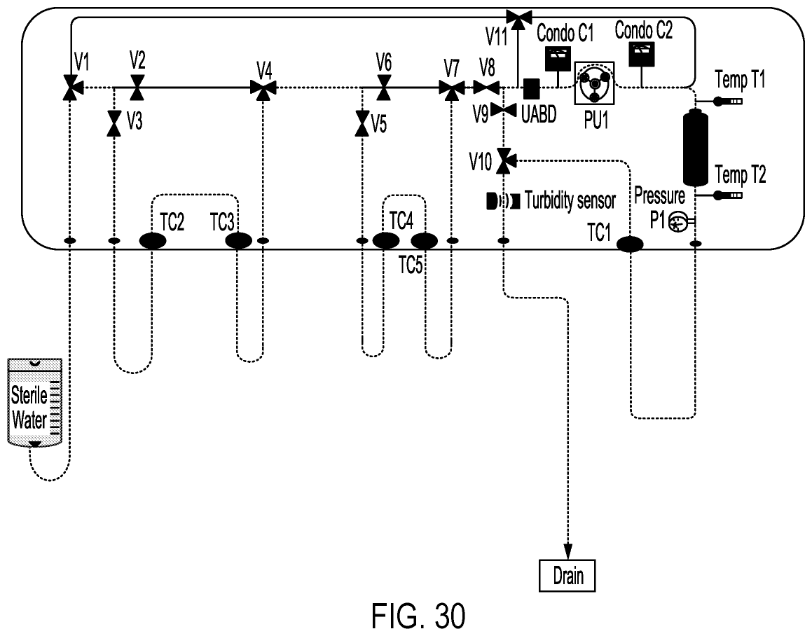

FIG. 30 discloses the source of sterile water would be used to circulate through the flow-through circuits created in FIG. 29 in order to flush the circuits of dialysate from the previous treatment.

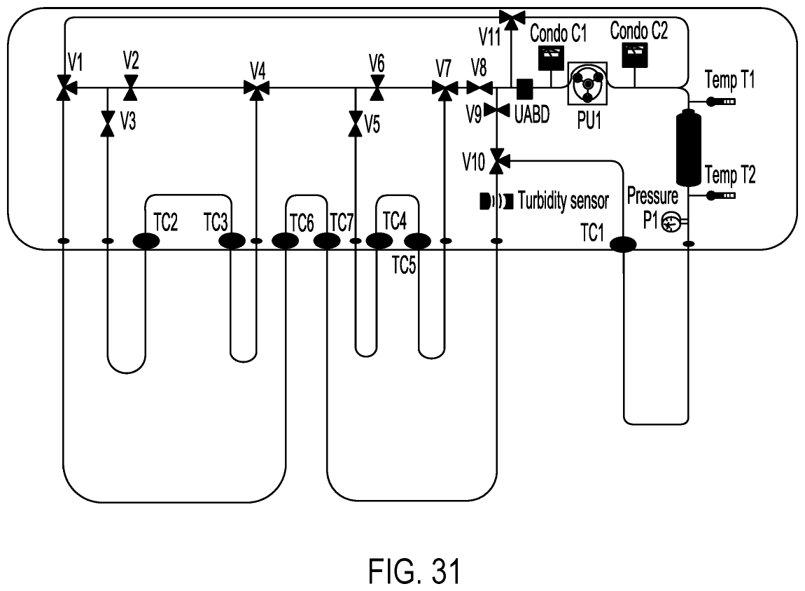

FIG. 31 discloses how the sterile water supply line and the drain line would be connected into the instrument in order to form a flow-through circuit which will allow hot water to be recirculated through all fluid pathways in order to accomplish disinfection.

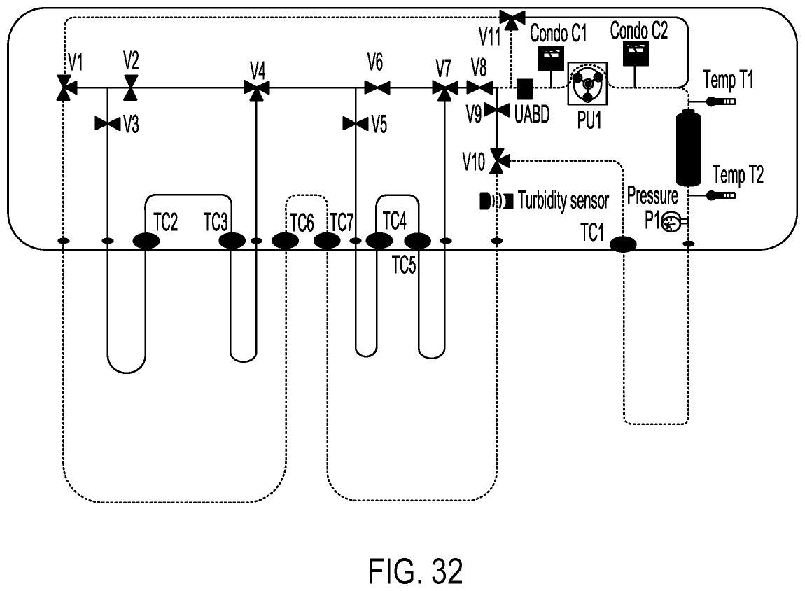

FIG. 32 discloses a graphic depiction of one of the disinfection modes illustrating how hot water would be recirculated through various of the fluid paths in order to accomplish disinfection.

This concept is illustrated in FIG. 22. The shaded rectangle represents the enclosure so the tubing that is shown inside that blue rectangle would be inside the instrument and the tubing and supply bags that are outside the rectangle would be outside the instrument.

The material of construction of the tubing that is inside the instrument would be inert, biocompatible, and heat resistant. Examples would include PVDF and FEP. This tubing would not be handled by the patient so it would not need to be flexible.

The tubing that is outside the instrument does need to be flexible because the patient will need to extract it from the machine at the initiation of a treatment and connect it to either supply bags, their transfer set (catheter), and possibly a drain bag. But these tubing segments must also be heat resistant and biocompatible since they will also be included in the disinfection process. Consequently, suitable materials for these segments would be silicone such as Sani-Tech® STHT®-C.

The reason these segments of tubing, as well as all other fluid contacting components, need to be heat resistant is that the preferred method of disinfecting the entire fluid path is by circulating water that is heated to at least 80° C. for at least one hour. This is a method that achieves “high level disinfection” which is a defined term that is accepted by the FDA for among others, the reprocessing of hemodialyzers and is recognized to kill all vegetative organisms. It is also the method used in a predicate device described, for example in U.S. Pat. No. 5,591,344.

Whereas, the fluid path could be disinfected by other means such as with chemical disinfectants (e.g. a mixture of peracetic acid and hydrogen peroxide, bleach), ultraviolet light, ozonated water, and other methods known in the art, heat is uniquely suited to this purpose since heat conducts through surfaces and can reach bacteria where they may have sequestered themselves away from contact by chemical disinfectants or other disinfection means as, for example, in the cracks or crevices in fluid paths constructed from tubing that is connected to other pieces of tubing with couplers or with other sensors and components used in a particular device. Also, this method avoids the cost of chemical disinfectants and the need for patients to handle and be exposed to them. The automated extended use of a dialysate fluid path (as opposed to a single use disposable) used in the performance of peritoneal dialysis has never before been described.

Where the internal tubing terminates at the face of the enclosure, it must mate with its corresponding external tubing. In order to minimize any crevices in the fluid path where bacteria may be able to be shielded from disinfectants, tubing couplers which minimize or eliminate dead spaces in the fluid path are desired. An example is the AVS Dead Space free tubing coupler manufactured by the AVS Romer GmbH & Co. Grafenau, Germany.

Romer also makes two and three way valves, such as the EAV 800, with the same dead space free construction.

In order to accomplish the inter-treatment disinfection process, a source of water is required. This can be accomplished by attaching a bag of sterile water in the same manner as the dialysate supply bags are attached (FIG. 23). Such bags of sterile water are readily available from several vendors.

At the end of a heat disinfection and cool-down cycle, and before the instrument is ready to initiate the next patient treatment, the sterile water from the previous disinfection cycle must be replaced with fresh dialysate from newly connected supply bags. This requirement dictates that the patient connection line be connected to the drain line so that the water contained therein can be flushed to drain and filled with dialysate. To accomplish that, a receptacle is designed into the face of the enclosure into which the patient transfer set connector is mated and locked in place (FIG. 24). The system shown in FIG. 24 may include an in-line heater such as the White Knight ultrapure PVDF/PVA fluid heater model FH1-FH7, a non-invasive conductivity sensor such as the Great Lakes Instruments GLI 3046C1T Electrodeless Conductivity Sensor, an ultrasonic air/bubble sensor such as the Introtek AD8/AD9 series, and an in-line pressure sensor with biocompatible and heat resistant materials of construction such as the Ashcroft® ZL91.

This receptacle (TC1) is designed so that hot water can circulate around the outside of the first few millimeters of the patient connector as well as through the lumen so that if any microorganisms were transferred to that surface by the patient through touch contamination, they would be killed by the disinfection process. It is also designed such that once the patient connector is fully inserted, the receptacle TC1 can be manipulated to lock the patient connector in place so that it cannot be accidentally dislodged.

The cycler instrument must contain a pump that is used to convey dialysate to and from the patient and also to recirculate water and dialysate through the fluid pathways according to the various modes of operation. This pump (PU1), could be of many different types including, but not limited to, roller peristaltic, linear peristaltic, mechanical or pneumatic diaphragm, axial flow, gear, and centrifugal.

In order to heat the dialysate to body temperature and also to heat the water to disinfection temperature, a heater is required. In this case, however, since the fluid pathways are mostly semi-permanent, an in-line heater can be employed. In order to control the temperature accurately in a feedback loop manner, temperature sensors such as thermistors are located just pre and post heater element.

Since it will be necessary to detect when all of the fluid in the fluidic circuit is either all water or all dialysate, a conductivity sensor (C1) is added. This sensor could be of many different designs and could invade the fluid path or be non-invasive such as the Great Lakes Instruments GLI 3046C1T Electrodeless Conductivity Sensor.

At times, such as when the instrument is first put into use or when a patient is traveling with it, the fluidic pathway may be air-filled. In those cases, it is necessary to first prime all of the air out of the circuit, replacing it with fluid since injecting air into a patient must be prevented. It is also possible during a treatment that one or more of the supply bags runs dry also allowing air to be brought into the fluid path. In order to determine when all air has been removed from the circuit during priming and also to detect an empty bag, an air/bubble sensor is placed just pre-pump. In that position, once air is detected during a treatment, the pump can be stopped and reversed thereby sending the air back into the bag from whence it came after which the appropriate valve (e.g. V3 is bag #1 is empty) can be closed and further air injection prevented. The sensors typically used for this purpose are ultrasonic such as the Introtek AD8/AD9 series shown below. The air alternately may be directed down the drain line past valve 212I.

It is necessary to insure that, when using a pump to instill or drain dialysate from a patient, the positive and negative pressure created never exceed safe limits. That is assured by incorporating a pressure sensor in the fluid path closest to the patient (P1). The pressure readings from this sensor are continuously fed back to the pump to control its speed so that the pressure on the patient's peritoneal cavity remains in a safe range. Once again, since the fluid path is semi-permanent and will be disinfected between each treatment, this pressure sensor can be designed as part of the fluid path as opposed to the non-invasive versions used by most other cyclers that involve the use of a diaphragm which is contiguous with the fluid path that transduces the fluid path pressure to an air column which is in contact with the pressure transducer. An example of an in-line pressure transducer that is made from inert, biocompatible material and is heat resistant is the Ashcroft® ZL91.

To initiate a treatment, a patient would first connect new bags of dialysate and sterile water to their respective supply lines that are attached to the instrument and either connect the drain line to a drain bag or locate it in an open drain. Once that is complete, the patient would initiate the priming sequence by selecting the appropriate indicator on a graphic user interface (GUI) which is either integral to the machine or wirelessly connected to it. This will instruct the software controlling the instrument to begin purging all fluid paths of water by running the pump and opening the appropriate valves to draw dialysate from all attached dialysate supply containers as shown in FIG. 25. The fluid path segment running through valve V9 will be primed during the initial patient drain.

The priming mode is terminated once the conductivity stabilizes at the value associated with the prescribed composition of dialysate. At that point, the GUI would indicate to the patient that it is time to disconnect the patient connection line from receptacle TC1 and connect it to their catheter/transfer set. Once done, the patient would so indicate to the GUI and the instrument would enter the initial patient drain mode as shown in FIG. 26.

In the performance of peritoneal dialysis it is very important to quantify the volume of fluid that enters and leaves the patient during every fill and drain cycle so that the net amount of fluid removed from the patient can be quantified and reported. This measurement must meet accuracy requirements which are typically more stringent than the typically pump can provide by, for example, multiplying stroke volume by the number of strokes. In order to accomplish this required accuracy while adding minimal complexity, cost and weight to the instrument, a novel and accurate method of measuring flow rate has been devised in this design.

Since a source of water already exists for the disinfection process and, since the difference in conductivity between water and peritoneal dialysate is great, a conductivity-time-of-flight (C-TOF) method for accurately determining flow rate can be implemented. This is accomplished by adding a conductivity cell (C2) on the opposite side of the pump from C1 and adding a water line from 3-way valve V1, through another 3-way valve (V11) and connecting into a 3-way tubing connector on the patient side of both the pump and C2 as shown in FIG. 27.

By rapidly opening and then closing valve V11, small pulses of water (e.g. 1 ml every 15 seconds) can be injected into the dialysate fluid path which will then pass C2 followed shortly by passing C1. By knowing the volume of the flow path between C2 and C1 and then determining the time elapsed between the trough of conductivity passing each conductivity sensor, the flow rate can be accurately calculated. This process could either be continuous (whenever the pump is moving fluid) or discontinuous where the C-TOF method is only used periodically to calibrate the pump stroke volume. If validation testing proves that the stroke volume remains mostly constant during a given treatment, then the pump-calibration method may prove sufficiently accurate.

The C-TOF method can also be implemented during the patient fill mode by adding another tubing segment between valve V11 and a 3-way tubing connector located on the dialysate supply bag side of C1 as shown in FIG. 28. Again, if the stroke volume calibration method is sufficiently accurate, implementing C-TOF on both sides of the pump may not be necessary.

Once the treatment is complete and the patient has received their last fill (if prescribed) from the optional and separate last fill bag, the patient would disconnect their patient connect line from their catheter/transfer set and reconnect it into TC1. They would then disconnect each dialysate supply bag one at a time. Since the supply bag lines are also designed to be semi-permanent and must be flushed and disinfected, a means for connecting them into a closed-loop recirculation circuit is necessary. To accomplish that, tubing connectors very similar, if not identical, to TC1 are implemented on the external surface of the instrument as shown in FIG. 29 (TC2-5). Behind those tubing connectors are tubing segments that connect each pair of supply line tubes to each other forming a flow-through circuit.

Once all dialysate supply line tubes are locked in place, the instrument can begin flushing the dialysate remaining in the fluidic pathway to drain (FIG. 30.). Multiple flush modes may be necessary in order to rinse all tubing segments. The flush mode is complete when the conductivity cells stabilize at the conductivity of sterile water.

Once all fluid paths have been adequately rinsed, the final two unconnected lines must be connected into a similar flow-through design such that hot water can be recirculated through all flow paths. This is accomplished by adding another pair of tubing connectors, TC6 and TC7, and connecting the water supply line and the drain line to them as shown in FIG. 31.

Once all fluid lines are inserted and locked into place, the circulation of hot water can be initiated. The temperature sensors will assure that all tubing segments are exposed to sufficient heat for a sufficient length of time to achieve high level disinfection as shown in FIG. 32.

Once high level disinfection has been achieved, the instrument will continue to recirculate the water, alternating through all fluid paths and will not allow the tubing to be removed until the temperature has cooled to at least 37 degrees centigrade. In order to assure that the tubing is sequestered so that it cannot be touched while it is hot, a door or cover will be designed to be closed over the tubing and locked. The instrument will incorporate a sensor on the door latch so that it will know when the door is locked in the closed position indicating that it is safe to initiate the heat sanitization cycle and it will not let the door be opened until the temperature has cooled to a safe level.

### EXAMPLE ENUMERATED EMBODIMENTS (EEES)

EEE 1 is a system for peritoneal dialysis, the system comprising: a housing; a water supply line, a dialysate supply line, a drain line, and a patient line each extending from the housing; a first port, a second port, a third port, and a fourth port that are accessible from outside the housing, wherein the first port is configured to connect to the water supply line, the second port is configured to connect to the dialysate supply line, the third port is configured to connect to the drain line, and the fourth port is configured to connect to the patient line; and a first flush line, a second flush line, and a third flush line each contained within the housing, wherein the first flush line terminates at the first port and at the third port, the second flush line terminates at the second port, and the third flush line terminates at the fourth port and at the drain line within the housing.

EEE 2 is the system of EEE 1, wherein the dialysate supply line is a first dialysate supply line, the system further comprising: a second dialysate supply line extending from the housing; and a fifth port accessible from outside the housing, wherein the fifth port is configured to connect to the second dialysate supply line, wherein the second flush line also terminates at the fifth port.

EEE 3 is the system of any one of EEEs 1-2, wherein the dialysate supply line is a first dialysate supply line, the system further comprising: a second dialysate supply line and a third dialysate supply line each extending from the housing; a fifth port and a sixth port each accessible from outside the housing, wherein the fifth port is configured to connect to the second dialysate supply line and the sixth port is configured to connect to the third dialysate supply line; and a fourth flush line contained within the housing, wherein the fourth flush line terminates at the fifth port and at the sixth port.

EEE 4 is the system of any one of EEEs 1-3, wherein the first port comprises a female connector comprising: an opening; an O-ring surrounding the opening; and one or more first lugs disposed around the opening, and the water supply line terminates at a male connector comprising: a stem having male threads on an exterior surface of the stem, wherein an interior surface of the stem defines a tube that is in fluid communication with the water supply line, wherein the male threads are configured to mate with female threads of an adapter for a bag containing supply dialysate; a shroud that surrounds a first portion of the stem such that a second portion of the stem extends distally beyond the shroud; a circumferential sealing surface proximal to the shroud; and one or more second lugs proximal to the circumferential sealing surface.

EEE 5 is the system of EEE 4, wherein the exterior surface is distally tapered.

EEE 6 is the system of any one of EEEs 4-5, wherein the male connector is connected to the water supply line at a proximal end of the male connector.

EEE 7 is the system of any one of EEEs 4-6, wherein the male connector is configured to be connected to the female connector by: inserting the male connector axially into the opening such that the one or more second lugs move past the one or more first lugs; and rotating the male connector such that the one or more second lugs move behind the one or more first lugs, thereby sealing the circumferential sealing surface against the O-ring and exposing the shroud to the first flush line.

EEE 8 is the system of EEE 7, wherein the female connector comprises: a sensing ring comprising one or more magnets, wherein the sensing ring is configured to rotate as the male connector is rotated within the opening; and a base comprising one or more Hall effect sensors, wherein the one or more Hall effect sensors are configured to indicate whether the male connector is properly connected to the female connector based on first rotational positions of the one or more magnets relative to the one or more Hall sensors.

EEE 9 is the system of any one of EEEs 1-8, wherein the fourth port comprises a female connector comprising: an opening; an O-ring surrounding the opening; and one or more first lugs disposed around the opening, and the patient line terminates at a male connector comprising: a stem having first interior surface that defines a tube that is in fluid communication with the patient line; a shroud that surrounds a first portion of the stem such that a second portion of the stem extends distally beyond the shroud, wherein the shroud comprises male threads on a second interior surface of the shroud that are configured to mate with female threads of an adapter for a catheter; a circumferential sealing surface proximal to the shroud; and one or more second lugs proximal to the circumferential sealing surface.

EEE 10 is the system of EEE 9, wherein an exterior surface of the stem is distally tapered.

EEE 11 is the system of any one of EEEs 9-10, wherein the male connector is connected to the patient line at a proximal end of the male connector.

EEE 12 is the system of any one of EEEs 9-11, wherein the male connector is configured to be connected to the female connector by: inserting the male connector axially into the opening such that the one or more second lugs move past the one or more first lugs; and rotating the male connector such that the one or more second lugs move behind the one or more first lugs, thereby sealing the circumferential sealing surface against the O-ring and exposing the shroud to the third flush line.

EEE 13 is the system of EEE 12, wherein the female connector comprises: a sensing ring comprising one or more magnets, wherein the sensing ring is configured to rotate as the male connector is rotated within the opening; and a base comprising one or more Hall effect sensors, wherein the one or more Hall effect sensors are configured to indicate whether the male connector is properly connected to the female connector based on first rotational positions of the one or more magnets relative to the one or more Hall effect sensors.

EEE 14 is the system of any one of EEEs 1-13, further comprising: one or more valves; and a pump.

EEE 15 is the system of EEE 14, wherein the system is configured to operate in a priming mode wherein: the patient line is connected to the fourth port; and the one or more valves are configured to allow the pump to draw supply dialysate through the dialysate supply line and force the supply dialysate through the patient line, the third flush line, and the drain line.

EEE 16 is the system of any one of EEEs 14-15, wherein the system is configured to operate in a patient drain mode wherein: the patient line is connected to a patient's peritoneum; and the one or more valves are configured to allow the pump to draw spent dialysate through the patient line and force the spent dialysate through the drain line.

EEE 17 is the system of any one of EEEs 14-16, wherein the system is configured to operate in a patient fill mode wherein: the patient line is connected to a patient's peritoneum; and the one or more valves are configured to allow the pump to draw supply dialysate through the dialysate supply line and force the supply dialysate through the patient line and into the patient's peritoneum.

EEE 18 is the system of any one of EEEs 14-17, wherein the system is configured to operate in a first stage of a flush mode wherein: the dialysate supply line is connected to the second port; the patient line is connected to the fourth port; and the one or more valves are configured to allow the pump to draw water through the water supply line and force the water through the patient line, the third flush line, and the drain line.

EEE 19 is the system of EEE 18, wherein the system is configured to operate in a second stage of the flush mode wherein: the dialysate supply line is connected to the second port; the patient line is connected to the fourth port; and the one or more valves are configured to allow the pump to draw water through the water supply line, the second flush line, and the fourth flush line, and force the water through the patient line, the third flush line, and the drain line.

EEE 20 is the system of EEE 19, further comprising a bypass line that connects the water supply line to the patient line, wherein the system is configured to operate in a third stage of the flush mode wherein: the dialysate supply line is connected to the second port; the patient line is connected to the fourth port; and the one or more valves are configured to allow the pump to draw water through the water supply line and the bypass line, and force the water through the patient line, the third flush line, and the drain line.

EEE 21 is the system of any one of EEEs 14-20, further comprising a heater, wherein the system is configured to operate in a first stage of a disinfect mode wherein: the water supply line is connected to the first port; the dialysate supply line is connected to the second port; the drain line is connected to the third port; the patient line is connected to the fourth port; and the one or more valves are configured to allow the pump to circulate water through the water supply line, the heater, the patient line, the third flush line, the drain line, and the first flush line, back to the water supply line, wherein the heater is configured to heat the water passing through the heater.

EEE 22 is the system of EEE 21, wherein the system is configured to operate in a second stage of the disinfect mode wherein: the water supply line is connected to the first port; the dialysate supply line is connected to the second port; the drain line is connected to the third port; the patient line is connected to the fourth port; and the one or more valves are configured to allow the pump to circulate water through the water supply line, the dialysate supply line, the second flush line, the fourth flush line, the heater, the patient line, the third flush line, the drain line, and the first flush line, back to the water supply line, wherein the heater is configured to heat the water passing through the heater.

EEE 23 is the system of EEE 22, further comprising a bypass line that connects the water supply line to the patient line, wherein the system is configured to operate in a third stage of the disinfect mode wherein: the water supply line is connected to the first port; the dialysate supply line is connected to the second port; the drain line is connected to the third port; the patient line is connected to the fourth port; and the one or more valves are configured to allow the pump to circulate water through the water supply line, the bypass line, the heater, the drain line, and the first flush line, back to the water supply line, wherein the heater is configured to heat the water passing through the heater.

EEE 24 is the system of any one of EEEs 14-23, further comprising a water supply bag and a disposable priming line, wherein the system is configured to operate in a first stage of an air removal mode wherein: the water supply line is connected to a first bag port of the water supply bag; the dialysate supply line is connected to the second port; the disposable priming line is connected to the first port and a second bag port of the water supply bag; the drain line is connected to the third port; the patient line is connected to the fourth port; and the one or more valves are configured to allow the pump to circulate water through the water supply line, the patient line, the third flush line, the drain line, the first flush line, and the disposable priming line, thereby trapping air received from the disposable priming line in the water supply bag.

EEE 25 is the system of EEE 24, wherein the system is configured to operate in a second stage of the air removal mode wherein: the water supply line is connected to a first bag port of the water supply bag; the dialysate supply line is connected to the second port; the disposable priming line is connected to the first port and a second bag port of the water supply bag; the drain line is connected to the third port; the patient line is connected to the fourth port; and the one or more valves are configured to allow the pump to circulate water through the water supply line, the dialysate supply line, the second flush line, the fourth flush line, the patient line, the third flush line, the drain line, the first flush line, and the disposable priming line, thereby trapping air received from the disposable priming line in the water supply bag.

EEE 26 is the system of EEE 25, further comprising a bypass line that connects the water supply line to the patient line, wherein the system is configured to operate in a third stage of the air removal mode wherein: the water supply line is connected to a first bag port of the water supply bag; the dialysate supply line is connected to the second port; the disposable priming line is connected to the first port and a second bag port of the water supply bag; the drain line is connected to the third port; the patient line is connected to the fourth port; and the one or more valves are configured to allow the pump to circulate water through the water supply line, the bypass line, the drain line, the first flush line, and the disposable priming line, thereby trapping air received from the disposable priming line in the water supply bag.

EEE 27 is the system of any one of EEEs 1-26, wherein the housing comprises an exterior storage surface, the system further comprising: a plurality of hooks disposed on the exterior heating surface, wherein the water supply line, the dialysate supply line, the drain line, and the patient line are configured to be wrapped around the plurality of hooks for storage during a disinfection procedure; and a lid configured to cover the exterior storage surface, the water supply line, the dialysate supply line, the drain line, and the patient line during the disinfection procedure.

EEE 28 is the system of EEE 27, further comprising a plurality of clips configured to hold the water supply line, the dialysate supply line, the drain line, and the patient line between the plurality of hooks.

EEE 29 is the system of any one of EEEs 27-28, wherein the system is configured to lock the lid such that the lid cannot be opened during the disinfection procedure.

EEE 30 is the system of EEE 1, further comprising a heater configured to heat a liquid that passes through the water supply line, the dialysate supply line, the drain line, the patient line, the first port, the second port, the third port, the fourth port, the first flush line, the second flush line, and the third flush line.

EEE 31 is a method of operating the system of any one of EEEs 1-30, the method comprising: making a determination that: (i) the water supply line is not connected to the first port, (ii) the dialysate supply line is not connected to the second port, (iii) the drain line is not connected to the third port, and (iv) the patient line is connected to the fourth port; and in response to making the determination, operating the system in the priming mode.

EEE 32 is a method of operating the system of any one of EEEs 1-30, the method comprising: making a determination that: (i) the water supply line is not connected to the first port, (ii) the dialysate supply line is not connected to the second port, (iii) the drain line is not connected to the third port, and (iv) the patient line is not connected to the fourth port; and in response to making the determination, operating the system in the patient drain mode.

EEE 33 is a method of operating the system of any one of EEEs 1-30, the method comprising: making a determination that: (i) the water supply line is not connected to the first port, (ii) the dialysate supply line is not connected to the second port, (iii) the drain line is not connected to the third port, and (iv) the patient line is not connected to the fourth port; and in response to making the determination, operating the system in the patient fill mode.

EEE 34 is a method of operating the system of any one of EEEs 1-30, the method comprising: making a determination that: (i) the water supply line is not connected to the first port, (ii) the dialysate supply line is connected to the second port, (iii) the drain line is not connected to the third port, and (iv) the patient line is connected to the fourth port; and in response to making the determination, operating the system in the flush mode.

EEE 35 is a method of operating the system of any one of EEEs 1-30, the method comprising: making a determination that: (i) the water supply line is connected to the first port, (ii) the dialysate supply line is connected to the second port, (iii) the drain line is connected to the third port, and (iv) the patient line is connected to the fourth port; and in response to making the determination, operating the system in the disinfect mode.

EEE 36 is the method of EEE 35, further comprising disabling the heater in response to determining that a threshold duration has passed while operating in the disinfect mode.

EEE 37 is a method of operating the system of any one of EEEs 1-30, the method comprising: making a determination that: (i) the disposable priming line is connected to the first port, (ii) the dialysate supply line is connected to the second port, (iii) the drain line is connected to the third port, and (iv) the patient line is connected to the fourth port; and in response to making the determination, operating the system in the air removal mode.

EEE 38 is a method of operating the system of any one of EEEs 1-30, the method comprising: operating the system in the priming mode while the dialysate supply line is connected to a dialysate supply bag and the patient line is connected to the fourth port, thereby filling the system with the supply dialysate; operating the system in the patient drain mode while the patient line is connected to the patient's peritoneum, thereby draining the spent dialysate from the patient's peritoneum through the drain line; operating the system in the patient fill mode while the patient line is connected to the patient's peritoneum and the dialysate supply line is connected to the dialysate supply bag thereby filling the patient's peritoneum with the supply dialysate; operating the system in the flush mode while the water supply line is connected to the water supply bag, the dialysate supply line is connected to the second port, and the patient line is connected to the fourth port, thereby filling the system with water; operating the system in the disinfect mode while the water supply line is connected to the first port, the dialysate supply line is connected to the second port, the drain line is connected to the third port, and the patient line is connected to the fourth port, thereby circulating the heated water through the first flush line, the second flush line, and the third flush line; and operating the system in the air removal mode while the water supply line is connected to a first bag port of the water supply bag, the dialysate supply line is connected to the second port, the disposable priming line is connected to the first port and a second bag port of the water supply bag, the drain line is connected to the third port, and the patient line is connected to the fourth port, thereby trapping air received from the disposable priming line in the water supply bag.

While various example aspects and example embodiments have been disclosed herein, other aspects and embodiments will be apparent to those skilled in the art. The various example aspects and example embodiments disclosed herein are for purposes of illustration and are not intended to be limiting, with the true scope and spirit being indicated by the following claims.

1. A system for peritoneal dialysis, the system comprising:
a housing;
a water supply line, a dialysate supply line, a drain line, and a patient line each extending from the housing;
a first port, a second port, a third port, and a fourth port that are accessible from outside the housing, wherein
the first port is configured to connect to the water supply line,
the second port is configured to connect to the dialysate supply line,
the third port is configured to connect to the drain line, and
the fourth port is configured to connect to the patient line; and
a first flush line, a second flush line, and a third flush line each contained within the housing, wherein
the first flush line terminates at the first port and at the third port,
the second flush line terminates at the second port, and
the third flush line terminates at the fourth port and at the drain line within the housing.

2. The system of claim 1, wherein the dialysate supply line is a first dialysate supply line, the system further comprising:
a second dialysate supply line extending from the housing; and
a fifth port accessible from outside the housing, wherein the fifth port is configured to connect to the second dialysate supply line,
wherein the second flush line also terminates at the fifth port.

3. The system of claim 1, wherein the dialysate supply line is a first dialysate supply line, the system further comprising:
a second dialysate supply line and a third dialysate supply line each extending from the housing;
a fifth port and a sixth port each accessible from outside the housing, wherein the fifth port is configured to connect to the second dialysate supply line and the sixth port is configured to connect to the third dialysate supply line; and
a fourth flush line contained within the housing, wherein the fourth flush line terminates at the fifth port and at the sixth port.

4-14. (canceled)

15. The system of claim 1, further comprising one or more valves and a pump, wherein the system is configured to operate in a priming mode wherein:
the patient line is connected to the fourth port; and
the one or more valves are configured to allow the pump to draw supply dialysate through the dialysate supply line and force the supply dialysate through the patient line, the third flush line, and the drain line.

16. The system of claim 1, further comprising one or more valves and a pump, wherein the system is configured to operate in a patient drain mode wherein:
the patient line is connected to a patient's peritoneum; and
the one or more valves are configured to allow the pump to draw a spent dialysate through the patient line and force the spent dialysate through the drain line.

17. The system of claim 1, further comprising one or more valves and a pump, wherein the system is configured to operate in a patient fill mode wherein:
the patient line is connected to a patient's peritoneum; and
the one or more valves are configured to allow the pump to draw supply dialysate through the dialysate supply line and force the supply dialysate through the patient line and into the patient's peritoneum.

18. The system of claim 1, further comprising one or more valves and a pump, wherein the system is configured to operate in a first stage of a flush mode wherein:
the dialysate supply line is connected to the second port;
the patient line is connected to the fourth port; and
the one or more valves are configured to allow the pump to draw water through the water supply line and force the water through the patient line, the third flush line, and the drain line.

19. The system of claim 18, further comprising:
a fourth flush line contained within the housing,
wherein the system is configured to operate in a second stage of the flush mode wherein:
the dialysate supply line is connected to the second port;
the patient line is connected to the fourth port; and
the one or more valves are configured to allow the pump to draw water through the water supply line, the second flush line, and the fourth flush line, and force the water through the patient line, the third flush line, and the drain line.

20. The system of claim 19, further comprising a bypass line that connects the water supply line to the patient line, wherein the system is configured to operate in a third stage of the flush mode wherein:
the dialysate supply line is connected to the second port;
the patient line is connected to the fourth port; and
the one or more valves are configured to allow the pump to draw water through the water supply line and the bypass line, and force the water through the patient line, the third flush line, and the drain line.

21. The system of claim 1, further comprising a heater, one or more valves, and a pump, wherein the system is configured to operate in a first stage of a disinfect mode wherein:
the water supply line is connected to the first port;
the dialysate supply line is connected to the second port;
the drain line is connected to the third port;
the patient line is connected to the fourth port; and
the one or more valves are configured to allow the pump to circulate water through the water supply line, the heater, the patient line, the third flush line, the drain line, and the first flush line, back to the water supply line,
wherein the heater is configured to heat the water passing through the heater.

22. The system of claim 21, further comprising:
a fourth flush line contained within the housing,
wherein the system is configured to operate in a second stage of the disinfect mode wherein:
the water supply line is connected to the first port;
the dialysate supply line is connected to the second port;
the drain line is connected to the third port;
the patient line is connected to the fourth port; and
the one or more valves are configured to allow the pump to circulate water through the water supply line, the dialysate supply line, the second flush line, the fourth flush line, the heater, the patient line, the third flush line, the drain line, and the first flush line, back to the water supply line,
wherein the heater is configured to heat the water passing through the heater.

23. The system of claim 22, further comprising a bypass line that connects the water supply line to the patient line, wherein the system is configured to operate in a third stage of the disinfect mode wherein:
the water supply line is connected to the first port;
the dialysate supply line is connected to the second port;
the drain line is connected to the third port;
the patient line is connected to the fourth port; and
the one or more valves are configured to allow the pump to circulate water through the water supply line, the bypass line, the heater, the drain line, and the first flush line, back to the water supply line,
wherein the heater is configured to heat the water passing through the heater.

24. The system of claim 1, further comprising one or more valves, a pump, a water supply bag, and a disposable priming line, wherein the system is configured to operate in a first stage of an air removal mode wherein:
the water supply line is connected to a first bag port of the water supply bag;
the dialysate supply line is connected to the second port;
the disposable priming line is connected to the first port and a second bag port of the water supply bag;
the drain line is connected to the third port;
the patient line is connected to the fourth port; and
the one or more valves are configured to allow the pump to circulate water through the water supply line, the patient line, the third flush line, the drain line, the first flush line, and the disposable priming line, thereby trapping air received from the disposable priming line in the water supply bag.

25. The system of claim 24, further comprising:
a fourth flush line contained within the housing,
wherein the system is configured to operate in a second stage of the air removal mode wherein:
the dialysate supply line is connected to the second port;
the drain line is connected to the third port;
the patient line is connected to the fourth port; and
the one or more valves are configured to allow the pump to circulate water through the water supply line, the dialysate supply line, the second flush line, the fourth flush line, the patient line, the third flush line, the drain line, the first flush line, and the disposable priming line, thereby trapping air received from the disposable priming line in the water supply bag.

26. The system of claim 25, further comprising a bypass line that connects the water supply line to the patient line, wherein the system is configured to operate in a third stage of the air removal mode wherein:
the dialysate supply line is connected to the second port;
the drain line is connected to the third port;
the patient line is connected to the fourth port; and
the one or more valves are configured to allow the pump to circulate water through the water supply line, the bypass line, the drain line, the first flush line, and the disposable priming line, thereby trapping air received from the disposable priming line in the water supply bag.

27-30. (canceled)

31. A method of operating the system of claim 1, the method comprising:
making a determination that: (i) the water supply line is not connected to the first port, (ii) the dialysate supply line is not connected to the second port, (iii) the drain line is not connected to the third port, and (iv) the patient line is connected to the fourth port; and
in response to making the determination, operating the system in a priming mode wherein:
the patient line is connected to the fourth port; and
one or more valves of the system are configured to allow a pump of the system to draw supply dialysate through the dialysate supply line and force the supply dialysate through the patient line, the third flush line, and the drain line.

32. A method of operating the system of claim 1, the method comprising:
making a determination that: (i) the water supply line is not connected to the first port, (ii) the dialysate supply line is not connected to the second port, (iii) the drain line is not connected to the third port, and (iv) the patient line is not connected to the fourth port; and
in response to making the determination, operating the system in a patient drain mode wherein:
the patient line is connected to a patient's peritoneum; and
one or more valves of the system are configured to allow a pump of the system to draw spent dialysate through the patient line and force a spent dialysate through the drain line.

33. A method of operating the system of claim 1, the method comprising:
making a determination that: (i) the water supply line is not connected to the first port, (ii) the dialysate supply line is not connected to the second port, (iii) the drain line is not connected to the third port, and (iv) the patient line is not connected to the fourth port; and
in response to making the determination, operating the system in a patient fill mode wherein:
the patient line is connected to a patient's peritoneum; and
one or more valves of the system are configured to allow a pump of the system to draw supply dialysate through the dialysate supply line and force the supply dialysate through the patient line and into the patient's peritoneum.

34. A method of operating the system of claim 1, the method comprising:
making a determination that: (i) the water supply line is not connected to the first port, (ii) the dialysate supply line is connected to the second port, (iii) the drain line is not connected to the third port, and (iv) the patient line is connected to the fourth port; and
in response to making the determination, operating the system in a flush mode wherein:
the dialysate supply line is connected to the second port;
the patient line is connected to the fourth port; and
one or more valves of the system are configured to allow a pump of the system to draw water through the water supply line and force the water through the patient line, the third flush line, and the drain line.

35. A method of operating the system of claim 1, the method comprising:
making a determination that: (i) the water supply line is connected to the first port, (ii) the dialysate supply line is connected to the second port, (iii) the drain line is connected to the third port, and (iv) the patient line is connected to the fourth port; and
in response to making the determination, operating the system in a disinfect mode wherein:
the water supply line is connected to the first port:
the dialysate supply line is connected to the second port;
the drain line is connected to the third port:
the patient line is connected to the fourth port; and
one or more valves of the system are configured to allow a pump of the system to circulate water through the water supply line, a heater of the system, the patient line, the third flush line, the drain line, and the first flush line, back to the water supply line,
wherein the heater is configured to heat the water passing through the heater.

36. (canceled)

37. A method of operating the system of claim 1, the method comprising:
making a determination that: (i) a disposable priming line of the system is connected to the first port, (ii) the dialysate supply line is connected to the second port, (iii) the drain line is connected to the third port, and (iv) the patient line is connected to the fourth port; and
in response to making the determination, operating the system in an air removal mode wherein:
the water supply line is connected to a first bag port of a water supply bag;
the dialysate supply line is connected to the second port;
the disposable priming line is connected to the first port and a second bag port of the water supply bag;
the drain line is connected to the third port;
the patient line is connected to the fourth port; and
one or more valves of the system are configured to allow a pump of the system to circulate water through the water supply line, the patient line, the third flush line, the drain line, the first flush line, and the disposable priming line, thereby trapping air received from the disposable priming line in the water supply bag.

38. A method of operating the system of claim 1, the method comprising:
operating the system in a priming mode while the dialysate supply line is connected to a dialysate supply bag and the patient line is connected to the fourth port, thereby filling the system with a supply dialysate;
operating the system in a patient drain mode while the patient line is connected to a patient's peritoneum, thereby draining a spent dialysate from the patient's peritoneum through the drain line;
operating the system in a patient fill mode while the patient line is connected to the patient's peritoneum and the dialysate supply line is connected to the dialysate supply bag thereby filling the patient's peritoneum with the supply dialysate;
operating the system in a flush mode while the water supply line is connected to a water supply bag, the dialysate supply line is connected to the second port, and the patient line is connected to the fourth port, thereby filling the system with water;
operating the system in a disinfect mode while the water supply line is connected to the first port, the dialysate supply line is connected to the second port, the drain line is connected to the third port, and the patient line is connected to the fourth port, thereby circulating the water through the first flush line, the second flush line, and the third flush line; and
operating the system in an air removal mode while the water supply line is connected to a first bag port of the water supply bag, the dialysate supply line is connected to the second port, a disposable priming line is connected to the first port and a second bag port of the water supply bag, the drain line is connected to the third port, and the patient line is connected to the fourth port, thereby trapping air received from the disposable priming line in the water supply bag.
